(19) United States
(12) Patent Application Publication
(10) Pub. No.: US 2026/0284247 A1
(43) Pub. Date: Sep. 24, 2026

(54) ULTRAVIOLET GENERATOR

(71) Applicant: Evoqua Water Technologies Limited, Caldicot (GB)

(72) Inventors: Richard JOSHI, Warrington (GB); George FOSTER, Manchester (GB); Thomas CROXSON, Blackburn (GB); James ELLIS, Stockport (GB); Liam DENNON, Chorley (GB)

(73) Assignee: Evoqua Water Technologies Limited, Caldicot, GB (GB)

(21) Appl. No.: 19/474,803

(22) PCT Filed: Apr. 12, 2024
(86) PCT No.: PCT/EP2024/060098

§ 371 Date: Oct. 13, 2025

Related U.S. Application Data
(60) Provisional application No. 63/459,289, filed on Apr. 14, 2023.
(60) Provisional application No. 63/531,843, filed on Aug. 10, 2023.

(51) Int. Cl. A61L 2/10; A61L 9/20; C02F 1/32

(52) U.S. Cl. CPC A61L 2/10; A61L 9/20; C02F 1/325; A61L 2202/11; A61L 2202/121; A61L 2202/122; A61L 2202/123; A61L 2209/12; C02F 2201/004; C02F 2201/3223; C02F 2201/3227; C02F 2303/04

(57) ABSTRACT

An ultraviolet (UV) generator includes: a tube forming a main body of the UV generator, where the tube includes titanium; a first end cap assembly coupled to a first end of the tube, where the first end cap assembly is formed of a first plurality of end cap portions; a second end cap assembly coupled to a second end of the tube opposite the first end, where the second end cap assembly is formed of a second plurality of end cap portions; and at least one UV lamp extending between the first end cap assembly and the second end cap assembly and positioned within an internal chamber of the tube.

### CROSS-REFERENCE TO RELATED APPLICATIONS

This application is a U.S. national phase of International Application No. PCT/EP2024/060098 filed Apr. 12, 2024, which claims the benefit of U.S. Provisional Patent Application Number 63/459,289 filed Apr. 14, 2023 and U.S. Provisional Patent Application Number 63/531,843 filed Aug. 10, 2023, the disclosures of which are hereby incorporated by reference in their entirety for all purposes.

### BACKGROUND OF THE INVENTION

### Field of the Invention

This invention relates generally to ultraviolet (UV) generators, as well as systems and methods including UV generators.

### Technical Considerations

UV light, such as from a UV generator, may be used in a purification system to inactivate or sterilize bacteria and break down contaminants contained in the fluid, such as in air and/or water. UV light may be capable of converting contaminants in water to carbon dioxide and water, or can convert halogenated compounds into halogenated acids. For example, a fluid, such as a liquid or gas, may be exposed to an effective dose of UV light radiation, i.e., a predetermined minimum intensity for a pre-determined minimum exposure time. This may include an inactivation of a target percentage of a target microorganism present in the fluid (e.g., more than 90%, or more than 95%, or more than 99%, or more than 99.99%). However, existing UV generators are limited by the environments.

As such, there is a current need for a more universal and cost-effective UV generator capable of being used in various environments and applications.

### SUMMARY OF THE INVENTION

In one aspect of the present invention, an ultraviolet (UV) generator includes: a tube forming a main body of the UV generator, where the tube includes titanium; a first end cap assembly coupled to a first end of the tube, where the first end cap assembly is formed of a first plurality of end cap portions; a second end cap assembly coupled to a second end of the tube opposite the first end, where the second end cap assembly is formed of a second plurality of end cap portions; and at least one UV lamp extending between the first end cap assembly and the second end cap assembly and positioned within an internal chamber of the tube.

At least one portion of each of the respective first and second end cap assemblies may be removable relative to one or more other portions of the respective first and second end cap assemblies. Each of the first end cap assembly and the second end cap assembly may be coupled to the tube by one or more fasteners. The tube includes at least one boss extending from an outer surface thereof, and where the at least one boss is configured to receive the at least one fastener. The UV generator may further include at least one O-ring seal positioned between at least one of the plurality of end cap portions of each of the first end cap assembly and the second end cap assembly. At least one of the first end cap assembly and the second end cap assembly includes: a first end cap portion coupled to the tube; a second end cap portion including an inlet/outlet opening and coupled to the first end cap portion; and a third end cap portion coupled to the second end cap portion. The inlet/outlet opening of the second end cap portion may be flush with the internal chamber of the tube. The third end cap portion may be removable relative to the first end cap portion and the second end cap portion. The third end cap portion may include at least one opening configured to allow the at least one UV lamp to be removed from the internal chamber of the tube through the at least one opening of the third end cap portion. The UV generator may further include at least one secondary tube including quartz, the at least one secondary tube configured to surround the at least one UV lamp and extend between the first end cap assembly and the second end cap assembly, and positioned within an internal chamber of the tube. At least one of the first end cap assembly and the second end cap assembly may be at least partially rotatable relative to the tube. The first end cap assembly may be rotated 90° relative to the second end cap assembly. The first end cap assembly may be rotated 180° relative to the second end cap assembly. The first end cap assembly may be formed of at least one plastic material and the second end cap assembly may be formed of at least one plastic material. At least one of the first end cap assembly and the second end cap assembly may be formed of at least one of high density polyethylene (HDPE), chlorinated polyvinyl chloride (CPVC), or polypropylene (PP). The UV generator may be configured to be mountable both vertically and horizontally. An interior surface of the tube may include an interior coating. The interior coating may include a titanium dioxide coating. The at least one UV lamp may include a plurality of UV lamps extending between the first end cap assembly and the second end cap assembly. The UV generator may further include a plurality of secondary tubes comprising quartz, wherein each secondary tube surrounds a respective one of the plurality of UV lamps and extends between the first end cap assembly and the second end cap assembly.

In another aspect of the present invention, a method includes: providing a UV generator including a first end cap and a second end cap, where the first end cap and second end cap are at a first relative alignment, and one of the first end cap and second end cap is fluidly connectable to a source of fluid to be treated; and providing instructions to adjust the first relative alignment to a second relative alignment by: removing at least one of the first end cap or second end cap from a tube of the UV generator; and securing to the tube the at least one of the first end cap or second end cap that was removed at a second relative alignment different from the first relative alignment.

The instructions may further include recoupling to the tube the at least one of the first end cap or second end cap that was removed at the second relative alignment. The instructions may further include rotating the at least one of the first end cap or second end cap relative to a central axis of the UV generator. The instructions may further include rotating the at least one of the first end cap or second end cap 90°relative to the central axis. The instructions may further include rotating the at least one of the first end cap or second end cap 180° relative to the central axis. At least one of the first end cap or second end cap may include an end cap assembly. The instructions may further include removing at least one fastener from at least one boss on the tube of the UV generator and from the end cap assembly. The instructions may further include removing a coupled second end cap portion and third end cap portion of the end cap assembly from the tube and from a first end cap portion of the end cap assembly. The instructions may further include recoupling the coupled second end cap portion and third end cap portion of the end cap assembly to the tube and to the first end cap portion in the second relative alignment. The method may further include instructions to install one or more UV lamps in the UV generator.

In another aspect of the present invention, a method includes: providing a UV generator comprising a first end cap and a second end cap, wherein at least one of the first end cap and second end cap are at least partially rotatable relative to a central axis of the UV generator, the first end cap is fluidly couplable to a first pipe, and the second end cap is fluidly couplable to a second pipe; and reconfiguring alignment of at least one of the first end cap and the second end cap by rotating at least one of the first end cap and the second end cap based on a position of at least one of the first pipe and the second pipe.

Various non-limiting examples and aspects of the present invention will now be described and set forth in the following numbered clauses:
Clause 1: An ultraviolet (UV) generator, comprising: a tube forming a main body of the UV generator, wherein the tube is comprised of titanium; a first end cap assembly coupled to a first end of the tube, wherein the first end cap assembly is formed of a first plurality of end cap portions; a second end cap assembly coupled to a second end of the tube opposite the first end, wherein the second end cap assembly is formed of a second plurality of end cap portions; and at least one UV lamp extending between the first end cap assembly and the second end cap assembly and positioned within an internal chamber of the tube.
Clause 2: The UV generator of clause 1, wherein at least one portion of each of the respective first and second end cap assemblies is removable relative to one or more other portions of the respective first and second end cap assemblies.
Clause 3: The UV generator of clause 1 or clause 2, wherein each of the first end cap assembly and the second end cap assembly are coupled to the tube by one or more fasteners.
Clause 4: The UV generator of clause 3, wherein the tube comprises at least one boss extending from an outer surface thereof, and wherein the at least one boss is configured to receive the at least one fastener.
Clause 5: The UV generator of any one of clauses 1-4, further comprising at least one O-ring seal positioned between at least one of the plurality of end cap portions of each of the first end cap assembly and the second end cap assembly.
Clause 6: The UV generator of any one of clauses 1-5, wherein at least one of the first end cap assembly and the second end cap assembly comprises: a first end cap portion coupled to the tube; a second end cap portion comprising an inlet/outlet opening and coupled to the first end cap portion; and a third end cap portion coupled to the second end cap portion.
Clause 7: The UV generator of clause 6, wherein the inlet/outlet opening of the second end cap portion is flush with the internal chamber of the tube.
Clause 8: The UV generator of clause 6 or clause 7, wherein the third end cap portion is removable relative to the first end cap portion and the second end cap portion.
Clause 9: The UV generator of any one of clauses 6-8, wherein the third end cap portion comprises at least one opening configured to allow the at least one UV lamp to be removed from the internal chamber of the tube through the at least one opening of the third end cap portion.
Clause 10: The UV generator of any one of clauses 1-9, further comprising at least one secondary tube comprised of quartz, the at least one secondary tube configured to surround the at least one UV lamp and extend between the first end cap assembly and the second end cap assembly, and positioned within an internal chamber of the tube.
Clause 11: The UV generator of any one of clauses 1-10, wherein at least one of the first end cap assembly and the second end cap assembly is at least partially rotatable relative to the tube.
Clause 12: The UV generator of clause 11, wherein the first end cap assembly is rotated 90° relative to the second end cap assembly.
Clause 13: The UV generator of clause 11, wherein the first end cap assembly is rotated 180° relative to the second end cap assembly.
Clause 14: The UV generator of any one of clauses 1-13, wherein the first end cap assembly is formed of at least one plastic material and the second end cap assembly is formed of at least one plastic material.
Clause 15: The UV generator of clause 14, wherein at least one of the first end cap assembly and the second end cap assembly are formed of at least one of high density polyethylene (HDPE), chlorinated polyvinyl chloride (CPVC), or polypropylene (PP).
Clause 16: The UV generator of any one of clauses 1-15, wherein the UV generator is configured to be mountable both vertically and horizontally.
Clause 17: The UV generator of any one of clauses 1-16, wherein an interior surface of the tube includes an interior coating.
Clause 18: The UV generator of clause 17, wherein the interior coating comprises a titanium dioxide coating.
Clause 19: The UV generator of any one of clauses 1-18, wherein the at least one UV lamp comprises a plurality of UV lamps extending between the first end cap assembly and the second end cap assembly.
Clause 20: The UV generator of clause 19, further comprising a plurality of secondary tubes comprising quartz, wherein each secondary tube surrounds a respective one of the plurality of UV lamps and extends between the first end cap assembly and the second end cap assembly.
Clause 21: A method, comprising: providing a UV generator comprising a first end cap and a second end cap, wherein the first end cap and second end cap are at a first relative alignment, and one of the first end cap and second end cap is fluidly connectable to a source of fluid to be treated; and providing instructions to adjust the first relative alignment to a second relative alignment by: removing at least one of the first end cap or second end cap from a tube of the UV generator; and securing to the tube the at least one of the first end cap or second end cap that was removed at a second relative alignment different from the first relative alignment.
Clause 22: The method of clause 21, further comprising instructions to install one or more UV lamps in the UV generator.
Clause 23: The method of clause 21 or clause 22, wherein the instructions further comprise: recoupling to the tube the at least one of the first end cap or second end cap that was removed at the second relative alignment.
Clause 24: The method of any one of clauses 21-23, wherein the instructions further comprise: rotating the at least one of the first end cap or second end cap relative to a central axis of the UV generator.
Clause 25: The method of clause 24, wherein the instructions further comprise: rotating the at least one of the first end cap or second end cap 90° relative to the central axis.

Clause 26: The method of clause 24, wherein the instructions further comprise: rotating the at least one of the first end cap or second end cap 180° relative to the central axis.

Clause 27: The method of any one of clauses 21-26, wherein at least one of the first end cap or second end cap comprises an end cap assembly.

Clause 28: The method of clause 27, wherein the instructions further comprise: removing at least one fastener from at least one boss on the tube of the UV generator and from the end cap assembly.

Clause 29: The method of clause 27 or clause 28, wherein the instructions further comprise: removing a coupled second end cap portion and third end cap portion of the end cap assembly from the tube and from a first end cap portion of the end cap assembly.

Clause 30: The method of clause 29, wherein the instructions further comprise: recoupling the coupled second end cap portion and third end cap portion of the end cap assembly to the tube and to the first end cap portion in the second relative alignment.

Clause 31: A method, comprising: providing a UV generator comprising a first end cap and a second end cap, wherein at least one of the first end cap and second end cap are at least partially rotatable relative to a central axis of the UV generator, the first end cap is fluidly couplable to a first pipe, and the second end cap is fluidly couplable to a second pipe; and reconfiguring alignment of at least one of the first end cap and the second end cap by rotating at least one of the first end cap and the second end cap based on a position of at least one of the first pipe and the second pipe.

### BRIEF DESCRIPTION OF THE DRAWINGS

The invention will be described with reference to the following drawing figures wherein like reference numbers identify like parts throughout.

FIG. 1 is a perspective view of a UV generator according to one aspect of the present disclosure;

FIG. 2A is a perspective view of a UV generator according to another aspect of the present disclosure;

FIG. 2B is a cross-sectional view of the UV generator of FIG. 2A along the line A;

FIG. 3A is a perspective view of a UV generator according to another aspect of the present invention;

FIG. 3B is a perspective view of a UV generator according to another aspect of the present invention;

FIG. 3C is a perspective view of a UV generator according to another aspect of the present invention;

FIG. 3D is a perspective view of a UV generator according to another aspect of the present invention;

FIG. 4 is a partial perspective view of a UV generator according to another aspect of the present invention;

FIG. 5A is a partial perspective view of a UV generator according to another aspect of the present invention;

FIG. 5B is a partial cross-sectional view of the UV generator of FIG. 5A along the line C;

FIG. 6 is a partial exploded view of a UV generator according to another aspect of the present invention;

FIG. 7 is a partial perspective view of a UV generator according to another aspect of the present invention;

FIG. 8 is a partial perspective view of a UV generator according to another aspect of the present invention;

FIG. 9 is a partial perspective view of a UV generator according to another aspect of the present invention;

FIG. 10A is a perspective view of a UV generator according to another aspect of the present invention;

FIG. 10B is a partial cross-sectional view of the UV generator of FIG. 10A along the line D;

FIG. 11 is a front view of a UV generator according to another aspect of the present invention;

FIG. 12 is a side view of a UV generator according to another aspect of the present invention ;

FIG. 13 is a perspective view of a UV generator according to another aspect of the present invention;

FIG. 14 is a partial exploded view of a UV generator according to another aspect of the present invention;

FIG. 15 is an exploded view of a quartz sealing arrangement according to another aspect of the present invention;

FIG. 16A is a perspective view of a UV generator according to another aspect of the present invention;

FIG. 16B is a cross-sectional view of the UV generator of FIG. 16A along the line E;

FIG. 17 is a front view of a UV generator according to another aspect of the present invention;

FIG. 18 is a side view of a UV generator according to another aspect of the present invention;

FIG. 19 is a partial exploded view of a UV generator according to another aspect of the present invention;

FIG. 20 is a partial exploded view of a UV generator according to another aspect of the present invention;

FIG. 21 is a partial exploded view of a UV generator according to another aspect of the present invention;

FIG. 22 is a partial exploded view of a UV generator according to another aspect of the present invention;

FIG. 23 is a partial exploded view of a UV generator according to another aspect of the present invention; and

FIG. 24 is a schematic view of a fluid inactivation/sterilization system according to another aspect of the present invention.

### DESCRIPTION OF THE PREFERRED EMBODIMENTS

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

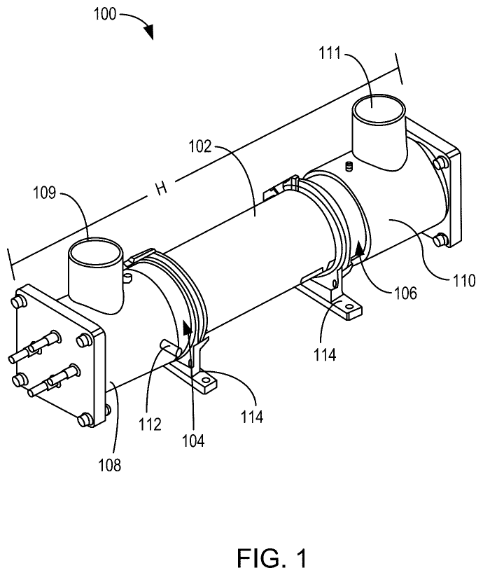

As shown in FIG. 1, a UV generator 100 is provided. The UV generator 100 may be configured to produce UV light that may be capable of killing, inactivating, or sterilizing microorganisms, and/or breaking down contaminants present in a fluid. As used herein, “UV light” refers to electromagnetic radiation having a wavelength in the range of from 100 nm to 400 nm. The UV generator 100 may be provided for the disinfection and/or chloramine reduction for a fluid. The UV generator 100 may be capable of providing a fluid with an effective dose of UV light. An “effective dose” of UV light refers to a pre-determined minimum intensity for a pre-determined minimum exposure time which can be determined from routine experimentation. The UV generator 100 may be configured to inactivate or sterilize a specified target percentage of a target microorganism in the fluid. For example, the UV generator 100 may be configured to inactivate or sterilize more than 90%, or more than 95%, or more than 99%, or more than 99.99% of a target microorganism in the fluid.

The UV generator 100 may include a tube 102. The tube 102 may form a main body of the UV generator 100. The tube 102 may be configured to protect components of the UV generator 100 contained within the tube 102. The tube 102 may be any size capable of protecting the components of the UV generator 100 positioned in the tube 102. While the tube 102 is shown as being cylindrical, this is just one such embodiment of the tube 102, such that the tube 102 may be any shape capable of protecting the components of the UV generator 100 positioned in the tube 102.

The tube 102 may include titanium (Ti). In contrast to other materials, a tube 102 including titanium has the benefit of producing a corrosion-resistant, high quality, lightweight, and high strength tube 102. Further, a tube 102 including titanium removes the need for both plastic (e.g., HDPE) and stainless steel versions of the chamber, thereby reducing product variation and increasing volumes per part. However, it is to be understood that the tube 102 may be formed of a material other than titanium. Minimal processing and/or machining is done to the tube 102, thereby keeping the complexity and costs related to manufacturing the tube relatively low.

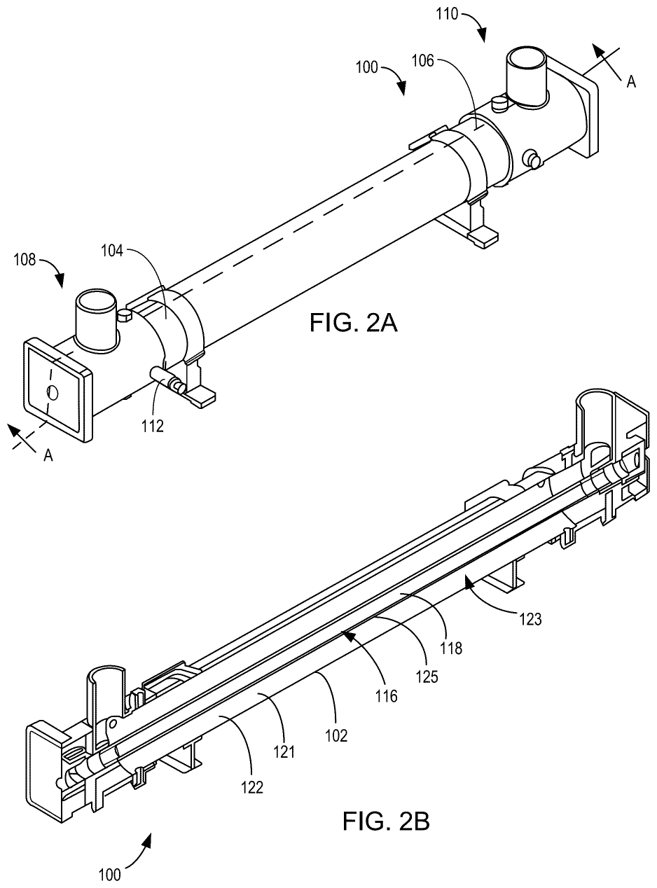

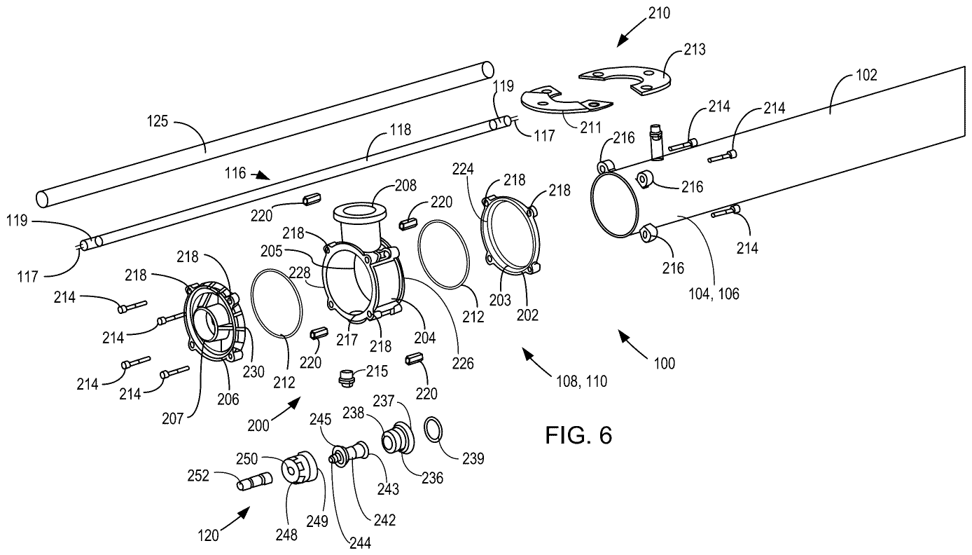

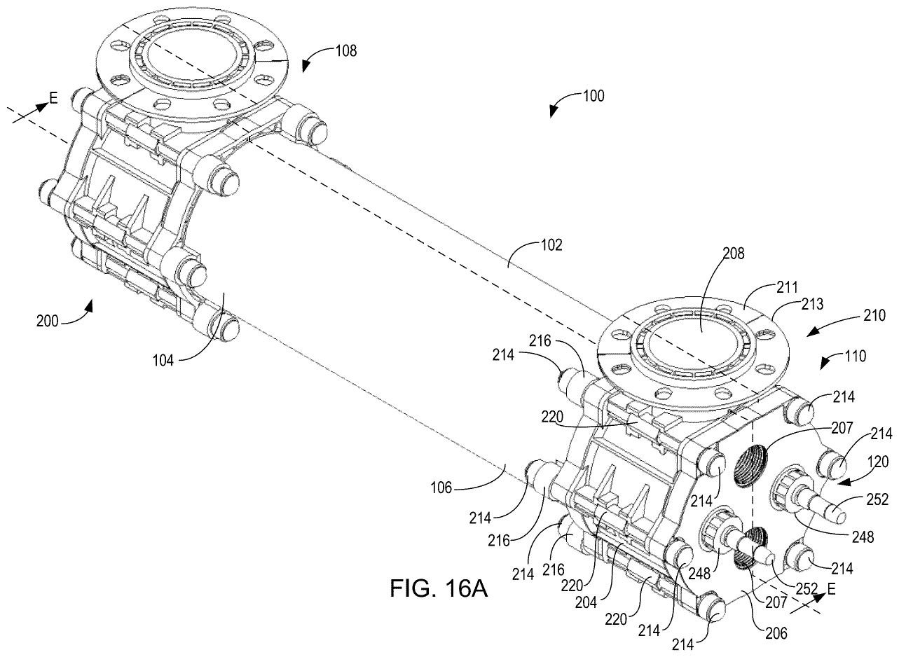

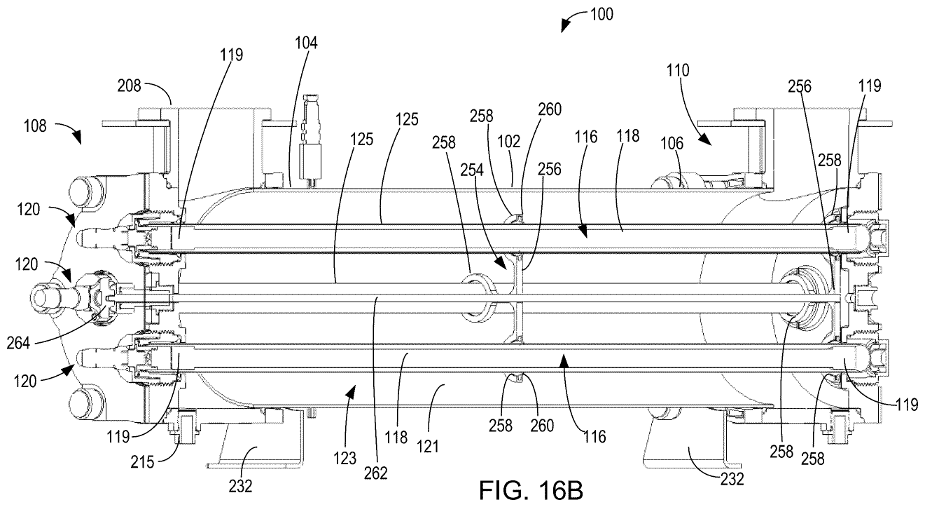

Referring to FIG. 2A-2B, the UV generator 100 may include a UV lamp 116. The UV lamp 116 may include a hollow conduit 118. The hollow conduit 118 may include a UV transparent material. For example, the hollow conduit 118 may include quartz. Quartz may be transparent to both UV light and visible light and have some physical properties similar to glass. The hollow conduit 118 may be sealed at both ends, with electrical connections 117 (see FIG. 6) extending through the seals 119 into the hollow conduit 118. The hollow conduit 118 may be filled with a gas known to produce UV light when excited with a sufficient electrical current from the electrical connections. Non-limiting examples of gasses that may fill the hollow conduit 118 include noble gases, such as argon, neon, xenon, and the like. As such, UV light is produced in the UV lamp 116 when the electrical current passes through the gas in the hollow conduit 118. In some non-limiting embodiments, the UV generator 100 may include a plurality of UV lamps 116, as shown in FIG. 16B.

The UV generator 100 may include a secondary tube 125. In some non-limiting embodiments, the secondary tube 125 may include a UV transparent material, such as quartz. The secondary tube 125 may have a liquid-tight seal. Thus, the secondary tube 125 may be positioned and configured to protect the UV lamp 116 and its electrical connections 117. In some non-limiting embodiments, the UV generator 100 may include a plurality of secondary tubes 125, as shown in FIG. 16B.

In some non-limiting embodiments, an internal surface 121 of the tube 102 may be treated to facilitate an advanced oxidation process (AOP) in combination with the UV light to increase the efficacy of the fluid treatment. In some non-limiting embodiments, an internal surface 121 of the tube 102 may include an interior coating 122. For example, the interior coating 122 may comprise a titanium dioxide coating. In some embodiments, the interior coating 122 is a titanium dioxide coating that can provide a photo-catalytic effect when UV reacts on the titanium dioxide coating, generating a photo-catalytic oxidation reaction to oxidize and destroy organic pollutants. Non-limiting examples of anatase titanium dioxide coating and/or methods for the interior coating 122 are disclosed in U.S. Pat. No. 9,492,810 B2, the disclosure of which is hereby incorporated by reference in its entirety for all purposes.

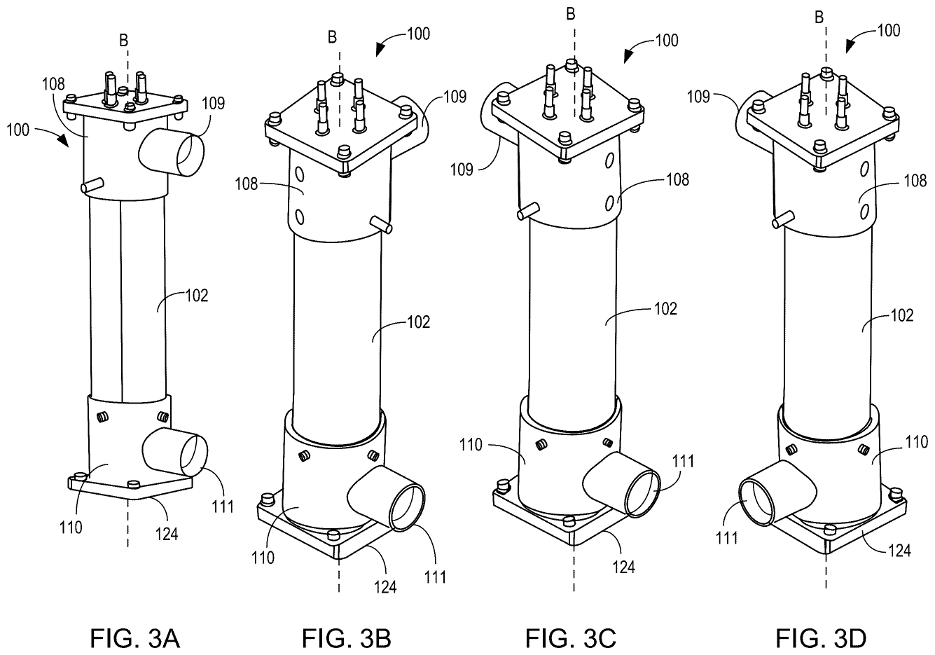

In some non-limiting embodiments, the UV generator 100 may be configured to be installed vertically or horizontally. For example, the UV generator 100 may be configured to be installed horizontally, as shown in FIGS. 1-2B. The UV generator 100 may include a pipe clip 114, such as a plurality of pipe clips 114. The pipe clip 114, such as a plurality of pipe clips 114, may be positioned on, such as positioned around, the tube 102, the first end cap 108, and/or the second end cap 110, and configured to mount and secure the UV generator 100 to a surface. The UV generator 100, in addition to or alternative to horizontal installation, may be configured to be installed vertically, as shown in FIGS. 3A-3D. If configured to be installed vertically, one or both of the first and second end caps 108, 110 may include a mounting foot surface 124. The mounting foot surface 124 on one or both of the first and second end caps 108, 110 may be configured to mount and secure the UV generator 100 to a surface.

The UV generator 100 may have a height H. The height H of the UV generator 100 may be dependent on the use of the UV generator 100 and/or the installation method. In some non-limiting embodiments, if the UV generator 100 is to be vertically-installed, the UV generator may have a height H of less than 2 m, or less than 1.5 m. For example, a vertically-installed UV generator 100 may have a height H in the range of from 0.5 m to 2.0 m, or from 0.75 m to 1.5 m.

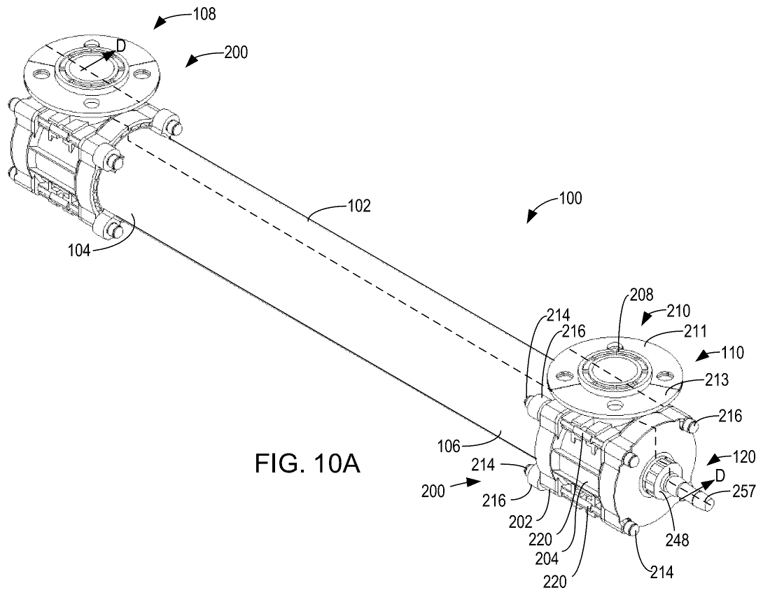

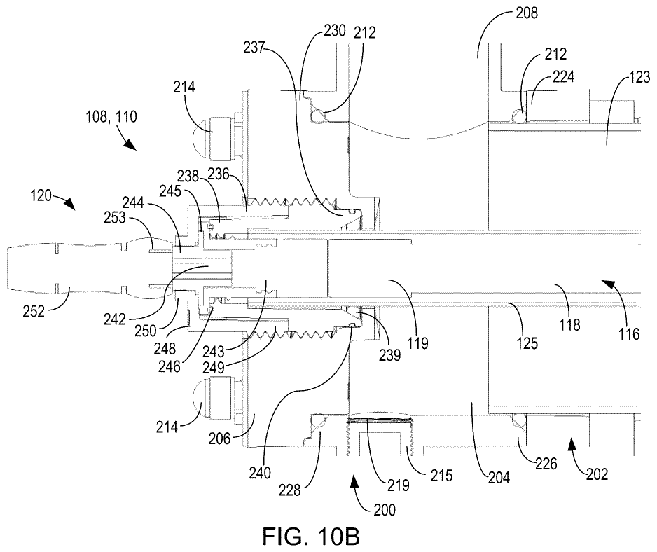

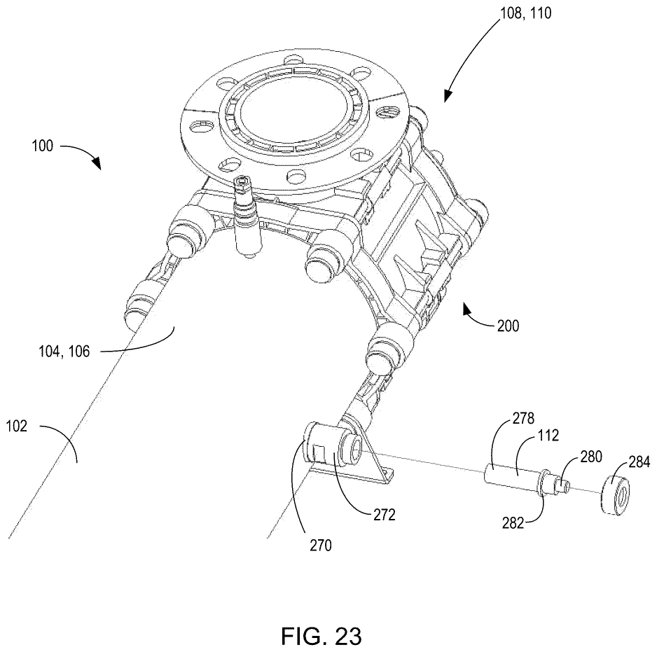

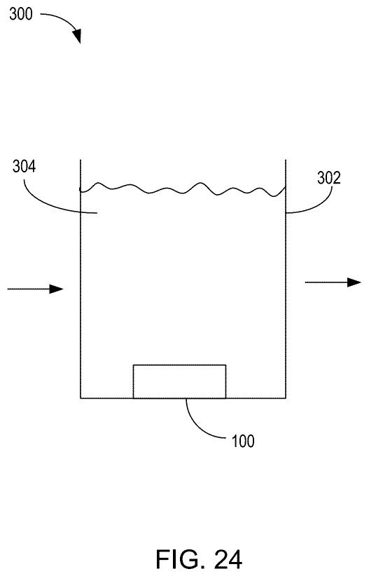

As shown in FIGS. 1, 10A, and 16A, the UV generator 100 may include a first end cap 108 coupled to a first end 104 of the tube 102 and a second end cap 110 coupled to a second end 106 of the tube 102. The end caps 108, 110 may each independently be a molded end cap or an end cap assembly 200. The end caps 108, 110 may be configured to house certain components, such as more complicated features and/or geometries, of the UV generator 100. For example, the end caps 108, 110 may house components such as, but not limited to, drain/vent ports, assembly/locating pins, sensor port(s), CIP port(s), etc. In one such example, the one or both of the end caps 108, 110 may be configured to receive a UV sensor 112. In some non-limiting embodiments, a UV sensor 112 may be positioned on the tube 102 as shown in FIGS. 23-24.

The first end cap 108 may include a first end cap inlet/outlet opening 109 and the second end cap 110 may include a second end cap inlet/outlet opening 111. The first end cap inlet/outlet opening 109 and the second end cap inlet/outlet opening 111 may each independently function as a fluid inlet or a fluid outlet for the UV generator 100. In this regard, one of the first end cap inlet/outlet opening 109 and the second end cap inlet/outlet opening 111 may function as an inlet, thereby supplying fluid to an internal chamber 123 of the tube 102 from an external fluid source, and the other of the first end cap inlet/outlet opening 109 and the second end cap inlet/outlet opening 111 may function as an outlet, thereby removing the fluid from the internal chamber 123 of the tube 102 back to the same or a different external fluid source. The internal chamber 123 of the tube 102 may be defined as the area inside of the tube 102 and outside of the secondary tube 125 (or hollow conduit 118 if secondary tube 125 is not present).

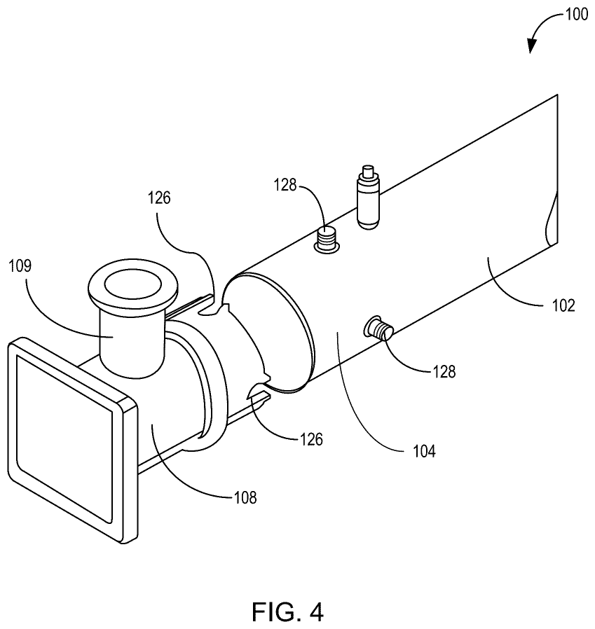

Referring to FIGS. 1-4, the end caps 108, 110 may be molded end caps. As used herein, “molded” end caps refer to end caps formed used one or more molding processes. Molded end caps may have the benefit of reducing the complexity and cost associated with producing the end caps 108, 110. The molded end caps 108, 110 may be substantially formed of a plastic material. Non-limiting examples of plastic materials that may be used to form the molded end caps 108, 110 include, but are not limited to, high density polyethylene (HDPE), chlorinated polyvinyl chloride (CPVC), polypropylene (PP), etc. A UV generator 100 that includes a tube 102 including titanium and end caps 108, 110 including HDPE/CPVC/PP produces a UV generator 100 of high strength, corrosion-resistant, and high quality. However, in alternative embodiments, the end caps 108, 110 may be substantially formed and/or constructed of one or more non-plastic materials, including, but not limited to, cast titanium.

Referring to FIGS. 3A-3D, the UV generator 100 provides multiple inlet/outlet process connection(s) and orientation options with the same parts. In FIG. 3A, the first end cap 108 and second end cap 110 are oriented such that the first end cap inlet/outlet opening 109 and second end cap inlet/outlet opening 111 are oriented the same direction; i.e., 0° relative to a central axis B of the UV generator 100. In FIG. 3B, one of the first end cap 108 or second end cap 110 may be rotated 90°, such that the first end cap inlet/outlet opening 109 and second end cap inlet/outlet opening 111 are oriented 90° from one another relative to the central axis B. In FIG. 3C, one of the first end cap 108 or second end cap 110 may be rotated 180°, such that the first end cap inlet/outlet opening 109 and second end cap inlet/outlet opening 111 are oriented 180° from one another relative to the central axis B. In FIG. 3D, one of the first end cap 108 or second end cap 110 may be rotated 270°, such that the first end cap inlet/outlet opening 109 and second end cap inlet/outlet opening 111 are oriented 270° (or 90° from the other direction) relative to the central axis B. In this regard, the UV generator 100 may be configured to allow the relative alignment of the first end cap 108 and second end cap 110 to be adjusted. Rotating of the first end cap 108 and/or second end cap 111 may be rotated clockwise or counterclockwise.

In this regard, in some non-limiting embodiments, the present invention also includes a method. The method may include a method to facilitate the treatment of a fluid. In some non-limiting embodiments, the method may include changing the relative alignment of the UV generator 100. The “relative alignment” of the UV generator 100 refers to the directional positioning of the inlet/outlet opening of one end cap compared to the directional positioning of the inlet/outlet opening of the other end cap relative to the central axis B. The ability to adjust the relative alignment of the UV generator 100 has the benefit of allowing the directional positioning of each of the end caps to be changed so that the inlet/outlet openings of the end caps can be connected to external piping. The present method of adjusting the relative alignment of the UV generator 100 allows for the quick adjustment of the directional positioning of each of the inlet/outlet openings of the end caps so that external piping may be easily connected. The method may include providing a UV generator 100 including a first end cap 108 and a second end cap 110. The first end cap 108 and second end cap 110 of the UV generator may be in a first relative alignment. The first relative alignment may be where the inlet/outlet opening 109 of the first end cap 108 is positioned 0°, or 90°, or 180°, or 270° (90° from the other direction), compared to the inlet/outlet opening 111 of the second end cap 110 relative to the central axis B, as is shown in FIGS. 3A-3D. One of the first end cap and second end cap may be fluidly connectable to a source of fluid to be treated.

The method may further include providing instructions to adjust the relative alignment of the UV generator 100, such as providing instructions to adjust the UV generator 100 from a first relative alignment to a second relative alignment. For example, the method may include providing instructions to adjust the relative alignment of the UV generator 100 by removing at least one of the first end cap 108 or second end cap 110 from a tube 102 of the UV generator 100; and securing to the tube 102 the at least one of the first end cap 108 or second end cap 110 that was removed into a second relative alignment different from the first relative alignment. The second relative alignment may be different from the first relative alignment. The second relative alignment may be where the inlet/outlet opening 109 of the first end cap 108 is positioned 0°, or 90°, or 180°, or 270° (90° from the other direction), relative to the inlet/outlet opening 111 of the second end cap 110. The instructions may further include providing instructions to removing at least one fastener 214 from the UV generator 100 and first end cap 108 or second end cap 110. For example, at least one fastener 214 may be used to couple the first end cap 108 or second end cap 110 to the UV generator 100 via at least one boss 216 on the tube 102 of the UV generator 100. The at least one fastener 214 may be removed such that the first end cap 108 or second end cap 110 may be removed from the UV generator 100, for example, by removing the at least one fastener 214 from the at least one boss 216 of the UV generator 100. The instructions may further include providing instructions to recouple the at least one of the first end cap 108 or second end cap 110 that was removed to the UV generator 100 by recoupling the at least one fastener 214 to the at least one of the first end cap 108 or second end cap 110 and to the at least one boss 216 of the UV generator 100. The instructions may further include providing instructions to rotate the at least one end cap that was removed relative to the central axis B before recoupling the removed end cap to the UV generator 100, such as providing instructions to rotate the at least one end cap that was removed 0°, or 90°, or 180°, or 270° relative to the central axis B before recoupling the removed end cap to the UV generator 100.

The method may further include following the instructions to adjust the UV generator 100 from a first relative alignment to a second relative alignment. The method may further include removing at least one of the first end cap 108 or second end cap 110 from the tube 102 of the UV generator 100. Removing at least one of the first end cap 108 or second end cap 110 may include removing at least one fastener 214 from the UV generator 100 and the first end cap 108 or second end cap 110, such as by decoupling the at least one fastener 214 from the at least one of the first end cap 108 or second end cap 110 and at least one boss 216 of the tube 102 of the UV generator 100. The method may further include rotating the at least one of the first end cap 108 or second end cap 110 that was removed relative to the central axis B, such as rotating the at least one of the first end cap 108 or second end cap 110 that was removed 0°, or 90°, or 180°, or 270° (90 ° from the other direction) relative to the central axis B. The method may further include securing to the tube 102 the at least one of the first end cap 108 or second end cap 110 that was removed at a second relative alignment different from the first relative alignment. The method may further include recoupling to the tube 102 the at least one of the first end cap 108 or second end cap 110 that was removed to the UV generator 100 in a second relative alignment that is different from the first relative alignment. Recoupling the at least one of the first end cap 108 or second end cap 110 that was removed to the UV generator 100 in a second relative alignment may include recoupling at least one fastener 214 to at least one boss 216 on the tube 102 of the UV generator 100 and to the at least one of the first end cap 108 or second end cap 110 that was removed.

In some non-limiting embodiments, the tube 102 may include at least one pin 128, such as a plurality of pins 128, extending outward from the external surface of the first end 104 and/or second end 106 of the tube 102. In some non-limiting embodiments, the tube 102 may include at least four pins 128 on each of the first end 104 and/or the second end 106. Alternatively, the tube 102 may include a different number of pins 128. In some non-limiting embodiments, the at least one pin 128, such as a plurality of pins 128, may be threaded. In some non-limiting embodiments, the first end cap 108 and/or second end cap 110 may include at least one pin opening 126, such as a plurality of pin openings 126, located on the external surface of the first end cap 108 and/or second end cap 110 and extending through the first end cap 108 and/or second end cap 110. The at least one pin opening 126, such as a plurality of pin openings 126, may be at least partially open so as to not form a complete shape, such that a pin 128 may be passed into the pin opening 126 and positioned therein. The pin openings 126 may be positioned on the first end cap 108 and/or second end cap 110 in positions corresponding to the positions of the pin(s) 128 on the first end 104 and/or second end 106 of the tube 102. Thus, each of the pins 128 present on the end of the tube 102 may pass through and into its corresponding pin opening 126 such that the pin(s) 128 are positioned in the pin opening 126 when the end cap 108, 110 is positioned on the first end 104 and/or second end 106 of the tube 102. A nut or other fastener may then be passed and secured onto the pin 128, thereby securing the end cap 108, 110 to the tube 102.

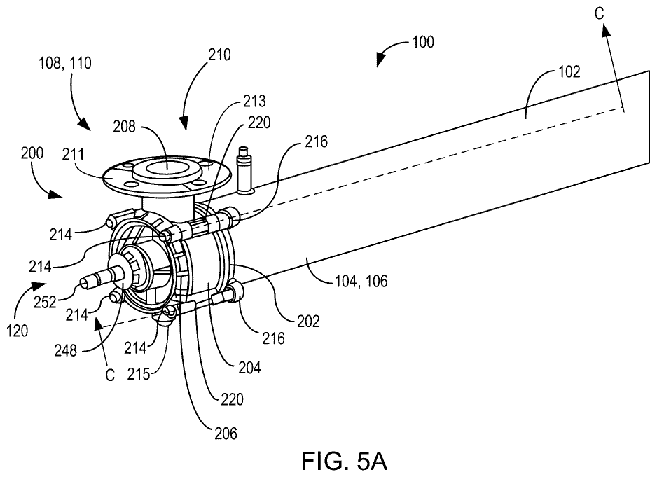

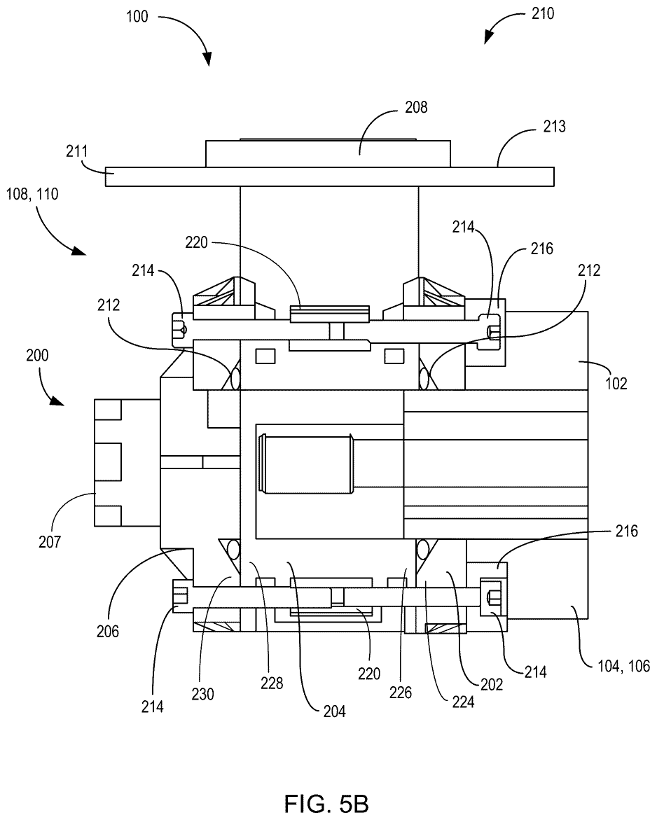

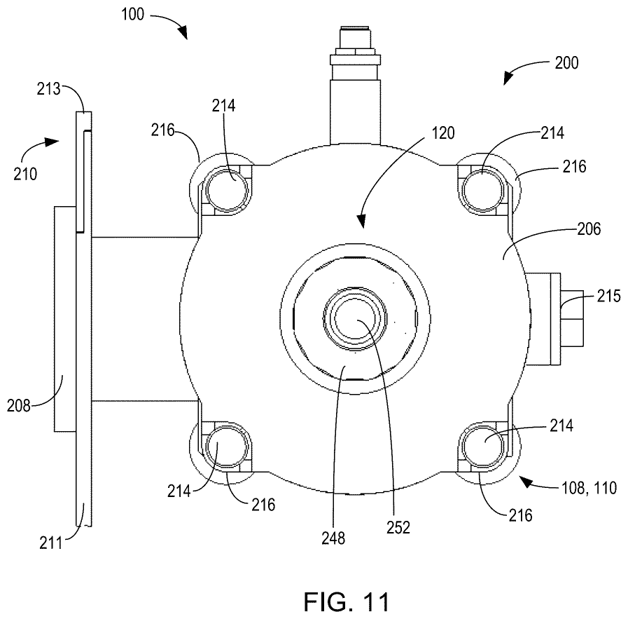

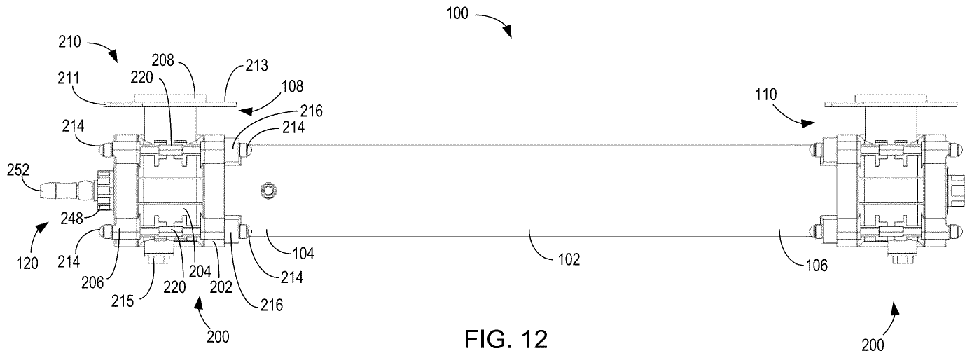

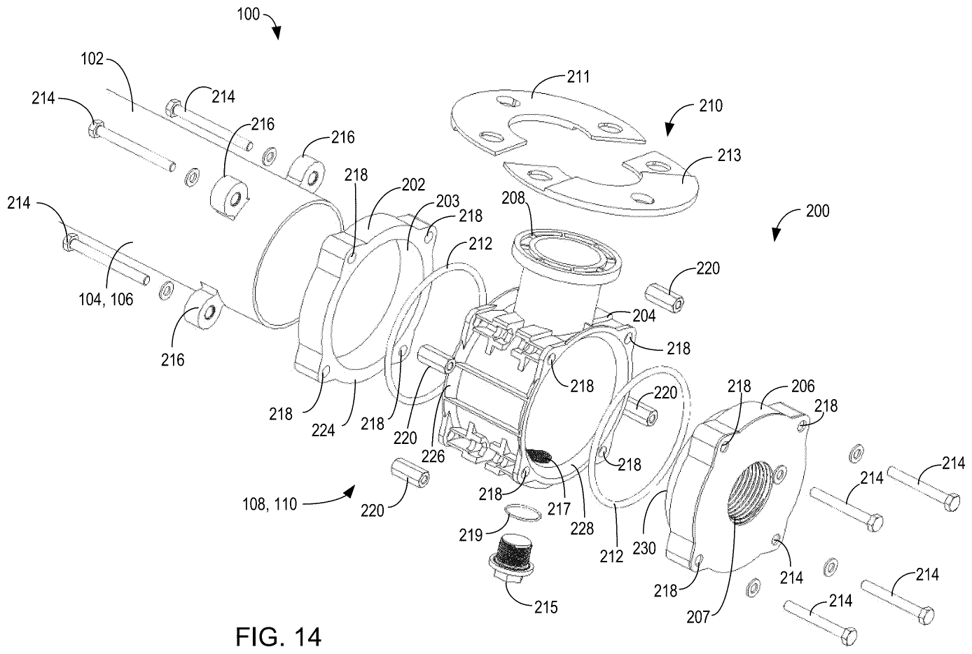

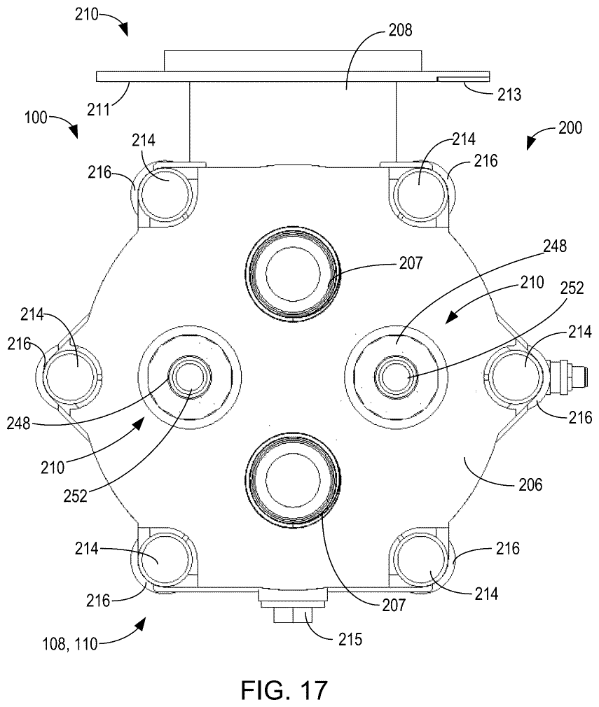

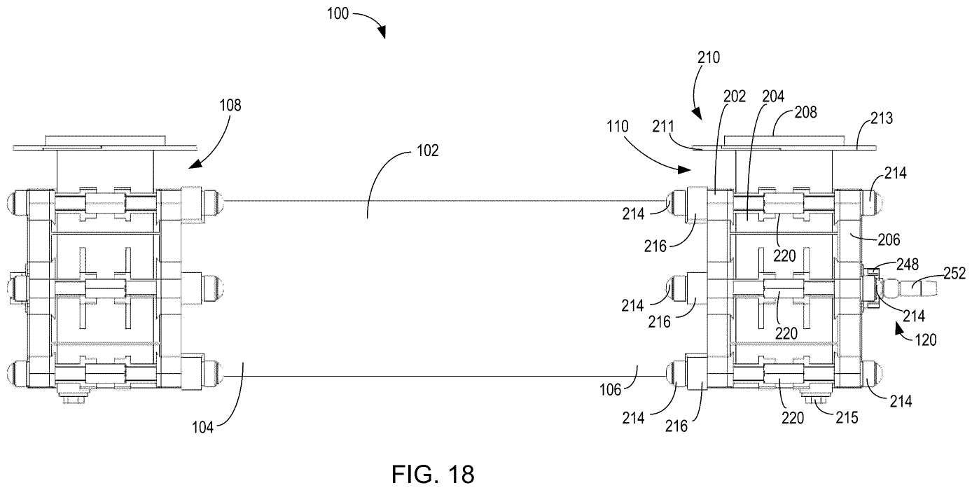

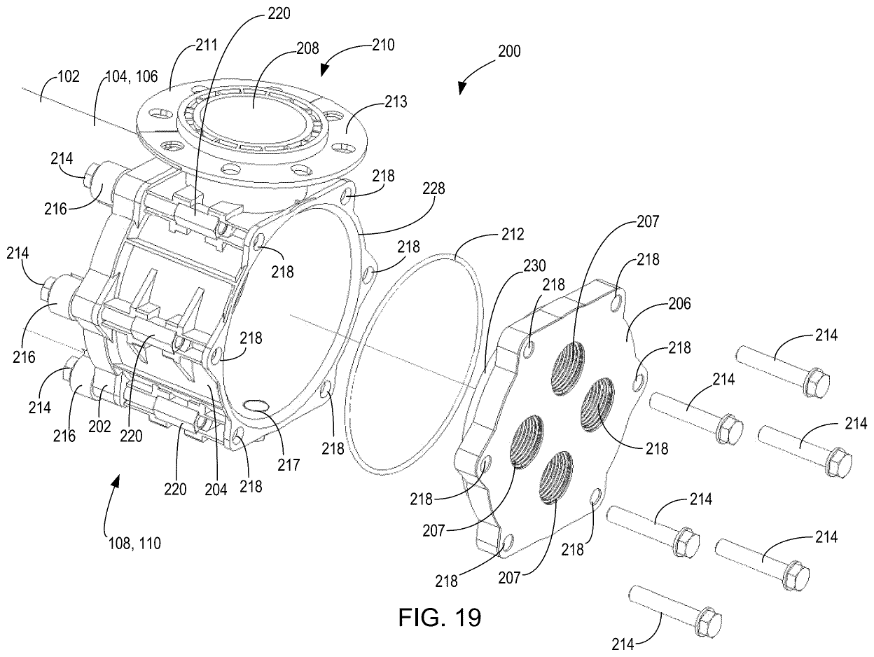

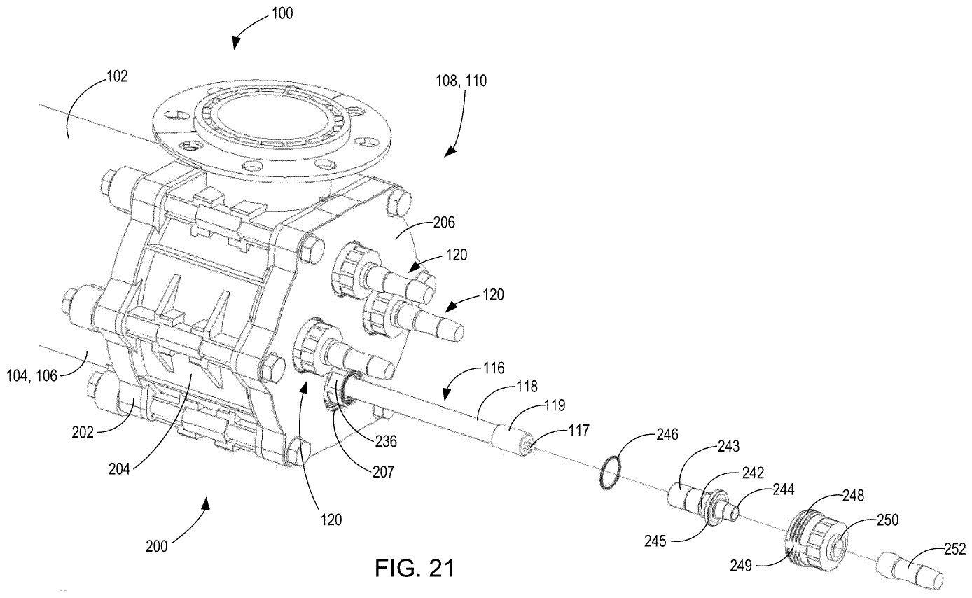

Referring to FIGS. 5A-6, 8-14 and 16A-24, in some non-limiting embodiments, the first end cap 108 and/or second end cap 110 may be an end cap assembly 200. The end cap assembly 200 may be capable of being at least partially removably affixed to respective end(s) 104, 106 of the tube 102. The end cap assembly 200 may include a plurality of end cap portions that in combination form the end cap assembly 200. For example, the end cap assembly may include at least one end cap portion, at least two end cap portions, or at least three end cap portions. At least one of the end cap portions of the end cap assembly may be removable relative to the other end cap portions. The end cap assembly 200 may include a first end cap portion 202, a second end cap portion 204, and a third end cap portion 206. While the end cap assembly 200 shown in the figures depicts an end cap assembly with three end cap portions, it is noted that the end cap assembly 200 can include a difficult combination and/or number of end cap portions, with different features present or absent on certain end cap portions, within the scope of the end cap assembly 200.

The end cap assembly 200, such as the first end cap portion 202, a second end cap portion 204, and a third end cap portion 206, may include a plastic material. For example, the first end cap portion 202, second end cap portion 204, and third end cap portion 206 may each independently include a plastic material, such as HDPE, PVC, PP, etc. In some non-limiting embodiments, the first end cap portion 202, second end cap portion 204, and third end cap portion 206 may each independently include a plastic material and/or other non-metallic material(s). Alternatively, the first end cap portion 202, second end cap portion 204, and third end cap portion 206 may each independently be substantially formed and/or constructed of one or more non-plastic materials (e.g., cast titanium).

The end cap assembly 200 may be positioned on either or both of the first end 104 and/or second end 106 of the tube 102. The first end cap portion 202, second end cap portion 204, and third end cap portion 206 may be positioned such that the first end cap portion 202 is positioned closest to the tube 102 and the third end cap portion 206 is positioned furthest from the tube 102 relative to the other portions of the end cap assembly 200. The first end cap portion 202 may be positioned closest to the tube 102 relative to the other portions of the end cap assembly 200. The first end cap portion 202 may have any shape such that the first end cap portion 202 is capable of being positioned on and/or over the first end 104 and/or second end 106 of the tube 102. For example, the first end cap portion 202 may be circular. The first end cap portion 202 may include an opening 203. The opening 203 of the first end cap portion 202 may have a circumference greater than the outer circumference of the tube 102. In this regard, the first end cap portion 202 may be positioned over at least a portion of the first end 104 and/or second end 106 of the tube 102 by positioning the first end 104 and/or second end 106 of the tube 102 within the opening 203 of the first end cap portion 202 such that the first end cap portion 202 rests on boss(es) 216. In some non-limiting embodiments, the end 224 of the first end cap portion 202 that is interfaced with the second end cap portion 204 may be tapered, such as tapered at a 45°angle. Alternatively, the end 224 of the first end cap portion 202 that is interfaced with the second end cap portion 204 may be flat, such that it is not tapered.

The second end cap portion 204 may be positioned further from the tube 102 compared to the first end cap portion 202, but closer to the tube 102 compared to the third end cap portion 206. The second end cap portion 204 may have any shape such that the second end cap portion 204 is capable of being positioned on and/or over the first end cap portion 202 and the first end 104 and/or second end 106 of the tube 102. For example, the second end cap portion 204 may be circular. The second end cap section 204 may include an opening 205. The opening 205 of the second end cap portion 204 may have a circumference greater than the outer circumference of the tube 102. In this regard, the second end cap portion 204 may be positioned over at least a portion of the first end 104 and/or second end 106 of the tube 102 by positioning the first end 104 and/or second end 106 of the tube 102 within the opening 205 of the second end cap portion 204. In another embodiment, the first end 104 and/or second end 106 of the tube 102 is not positioned within the opening 205 of the second end cap portion 204 such that the second end cap portion 204 is coupled to the first end cap portion 202 but does not house a portion of the tube 102. One or both ends 226, 228 of the second end cap portion 204 may be tapered, such as tapered at a 45° angle. Alternatively, one or both ends 226, 228 of the second end cap portion 204 may be flat, such that one or both ends 226, 228 are not tapered.

The second end cap portion 204 may include an inlet/outlet opening 208 extending from second end cap portion 204. The inlet/outlet opening 208 may function as a fluid inlet and/or a fluid outlet for the UV generator 100. In this regard, the inlet/outlet opening 208 may function as an inlet, thereby supplying fluid to an internal chamber 123 of the tube 102 from an external fluid source, and/or may function as an outlet, thereby removing the fluid from the internal chamber 123 of the tube 102 back to the same or a different external fluid source. Alternatively, the inlet/outlet opening 208 may be present on a different end cap portion. The inlet/outlet opening 208 may be located on the end cap assembly 200 such that it is flush with the internal chamber 123 of the tube 102. By having the inlet/outlet opening 208 flush with the internal chamber 123, the inlet/outlet opening 208 may operate without the need for an air valve.

The inlet/outlet opening 208 may include a retaining plate 210 configured to aid in securing the inlet/outlet opening 208 to another surface, for example, to provide fluid communication between the inlet/outlet opening 208 and another surface. In some non-limiting embodiments, the retaining plate 210 may include a first plate portion 211 and a second plate portion 213. The first plate portion 211 and second plate portion 213 may be coupled together, such as fastened together with a fastener. If the retaining plate 210 includes the first plate portion 211 and the second plate portion 213, the retaining plate may be coupled, such as fastened to the inlet/outlet opening 208 before or after mounting of the UV generator 100.

The second end cap portion 204 may include a drainage plug 215. The drainage plug 215 may correspond to a drainage hole 217 located on and through the outer surface of the second end cap portion 204. The drainage plug 215 may be configured to seal the drainage hole 217 when the drainage plug is positioned in the drainage hole 217, and allow fluid to flow out of the drainage hole 217 when the drainage plug 215 is removed from the drainage hole 217. In some non-limiting embodiments, the drainage plug 215 and the drainage hole 217 may be threaded. Alternatively, the drainage plug 215 and drainage hole 217 may be located on an end cap portion different from the second end cap portion 204. An o-ring seal 219 may be positioned around the drainage plug 215 and in the drainage hole 217 to aid in sealing the drainage hole 217.

The third end cap portion 206 may be positioned further away from the tube 102 compared to the first end cap portion 202 and second end cap portion 204. The third end cap portion 206 may have a shape such that the third end cap portion 206 is capable of being positioned on and/or over the first end cap portion 202 and second end cap portion 204. For example, the third end cap portion 206 may be circular.

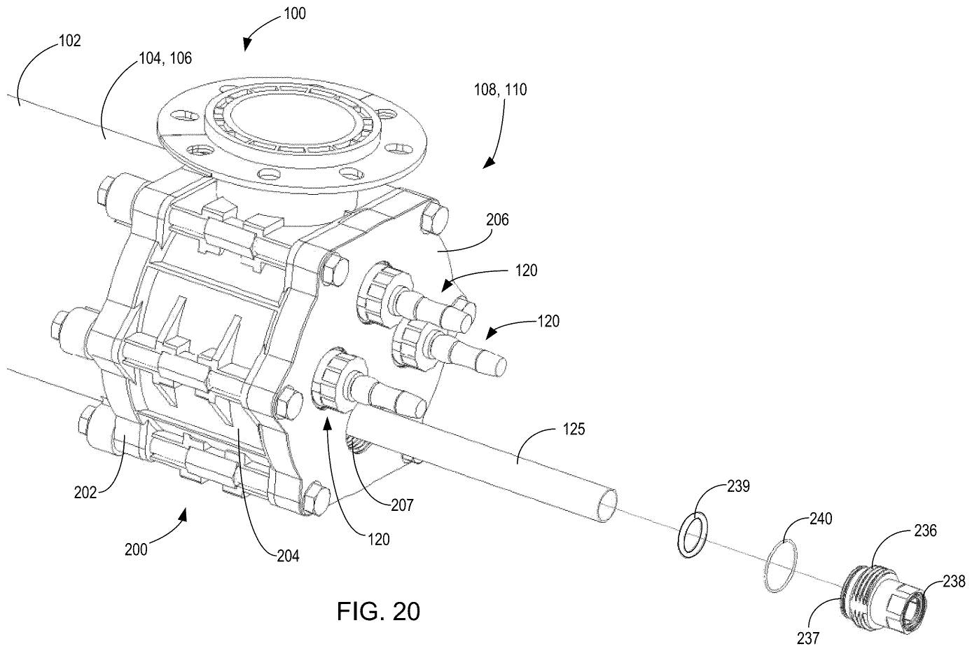

The third end cap portion 206 may include an opening 207. The opening 207 of the third end cap portion 206 may have a circumference less than the outer circumference of the tube 102, such that the third end cap portion 206 may be positioned on the first end cap portion 202 and second end cap portion 204, but cannot have the first end 104 or second end 106 of the tube 102 positioned within the opening 207 of the third end cap portion 206. The opening 207 of the third end cap portion 206 may have a circumference greater than the outer circumference of the UV lamp 116, such that the UV lamp 116 may be removed from the tube 102 and out the end cap assembly 200 via the opening 207 in the third end cap portion 206. The opening 207 of the third end cap portion 206 may have a circumference greater than the outer circumference of the secondary tube 125, such that the secondary tube 125 may be removed from the tube 102 and out the end cap assembly 200 via the opening 207 in the third end cap portion 206. The opening 207 of the third end cap portion 206 may be configured to receive and house the sealing arrangement 120. In some non-limiting embodiments, the opening 207 of the third end cap portion 206 may be threaded. The circumference of the opening 207 of the third end cap portion 206 may be stepped, such that the circumference of the opening 207 changes at different positions of the third end cap portion 206. The end 230 of the third end cap portion 206 that is interfaced with the second end cap portion 204 may be tapered, such as tapered at a 45°angle. Alternatively, the end 230 of the third end cap portion 206 that is interfaced with the second end cap portion 204 may be flat, such that the end 230 is not tapered. In some non-limiting embodiments, the third end cap portion 206 may include a plurality of openings 207, as shown in FIG. 20.

The end cap assembly 200 may be coupled to the first end 104 and/or the second end 106 of the tube 102. For example, the first end cap portion 202 may be coupled to the first end 104 and/or second end 106 of the tube 102, the second end cap portion 204 may be coupled to first end cap portion 202 (and optionally the first end 104 and/or second end 106 of the tube 102 if the tube 102 extends far enough into the opening 205 of the second end cap portion 204), and the third end cap portion 206 may be coupled to the second end cap portion 204.

In some non-limiting embodiments, the end cap assembly 200 may be fastened to the tube 102. For example, the first end cap portion 202 may be fastened to the tube 102, the second end cap portion 204 may be fastened to the first end cap portion 202, and the third end cap portion 206 may be fastened to the second end cap portion 204. Each of the first end cap portion 202, the second end cap portion 204, and the third end cap portion 206 may include at least one hole 218, such as a plurality of holes 218, extending from the outer surface of said portion 202, 204, 206 and configured to receive a fastener 214. The at least one hole 218, such as a plurality of holes 218, may be spaced along the outer circumference of each of first end cap portion 202, the second end cap portion 204, and the third end cap portion 206. For example, the at least one hole 218, such as the plurality of holes 218, may be located at the same position(s) along the outer circumference of each of the first end cap portion 202, the second end cap portion 204, and the third end cap portion 206, respectively, such that a hole 218 on each of the first end cap portion 202, the second end cap portion 204, and the third end cap portion 206 line up to receive the fastener 214. The hole 218, such as the plurality of holes 218, on each portion 202, 204, 206 may be threaded. In some non-limiting embodiments, the first end cap portion 202 may include at least four holes 218, the second end cap portion 204 may include at least eight holes 218 with four holes 218 positioned towards one end of the second end cap portion 218 and the other four holes 218 positioned towards the other end of the second end cap portion 218 and lining up with the first four holes 218, and the third end cap portion 206 may include at least four holes 218. Alternatively, each of the first end cap portion 202, the second end cap portion 204, and the third end cap portion 206 may include a different number of holes 218. The fastener(s) 214 may be any fastener capable of fitting in the hole(s) 218 and securing each portion of the end cap assembly 200 to each other and to the tube 102. For example, the fastener 214 may be a metallic fastener, such as a stainless steel A4 bolt 214.

The tube 102 may include at least one boss 216, such as a plurality of bosses 216. The boss(es) 216 may be positioned and spaced along the outer circumference of the tube 102 at the first end 104 and/or the second end 106 and in positions corresponding to the positions and spacing of the hole(s) 218 of the first end cap portion 202, the second end cap portion 204, and the third end cap portion 206. The boss(es) 216 of the tube 102 may be configured to each receive a fastener 214 and aid in fastening the end cap assembly 200 to the tube 102. In some non-limiting embodiments, the tube 102 may include at least four bosses 216. Alternatively, the tube 102 may include some other number of bosses 216.

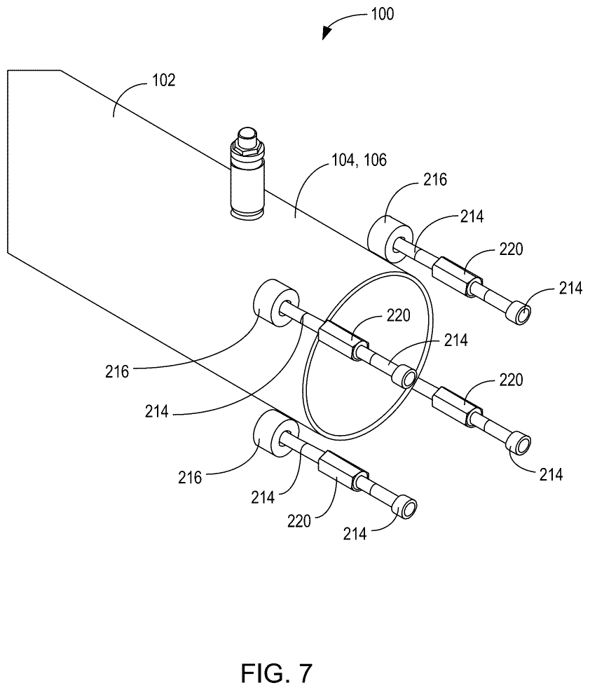

In some non-limiting embodiments, at least one nut 220, such as a plurality of nuts 220, may each be positioned on the outer circumference of the second end cap portion 204. The nut 220, such as plurality of nuts 220, may each be positioned such that one end of the nut is axially aligned with a hole 218 at one end of the second end cap portion 204 and the other end of the nut is axially aligned with a hole 218 at the other end of the second end cap portion 204. In this regard, a series of aligning holes 218 may be fastened by two fasteners 214. For example, a first fastener 214 may be positioned through a hole 218 of the first end cap portion 202, through a hole 218 of the second end cap portion 204, and with the end of the first fastener 214 positioned in the nut 220. A second fastener 214 may then be positioned through a hole 218 on the third end cap portion 206, through a hole on the second end cap portion 204, and with the end of the second fastener 214 positioned also in the nut 220. This arrangement is shown in FIG. 7, which has removed the end cap portions for clarity. Alternatively, a single fastener 214 may be used to fasten a series of aligning holes 218.

Similar to FIGS. 3A-3D, the end cap assembly 200 may be positioned on the first end 104 and/or second end 106 of the tube 102 such that the inlet-outlet opening is facing a specific direction. For example, relative to an inlet/outlet opening on an end cap at the other end of the tube 102, the end cap assembly 200 may be positioned on the first end and/or second end 106 such that the inlet/outlet opening 208 is oriented 0°, or 90°, or 180°, or 270° (90 ° from the other direction) compared to the inlet/outlet opening of the end cap at the other end of the tube 102. In this regard, the aforementioned method may include providing a UV generator 100 including a first end cap 108 and a second end cap 110, where each end cap may independently be an end cap assembly 200. If one or both of the first end cap 108 or second end cap 110 are an end cap assembly 200, the end cap assembly 200 may be removed, rotated, and/or recoupled to the UV generator 100 in the same or similar manner as if the end cap was not an end cap assembly 200.

The method may include providing instructions to adjust the relative alignment of the UV generator 100, such as providing instructions to adjust the UV generator 100 from a first relative alignment to a second relative alignment. For example, the method may include providing instructions to adjust the relative alignment of the UV generator 100 by removing at least one of the first end cap 108 or second end cap 110; and securing the at least one of the first end cap 108 or second end cap 110 that was removed into a second relative alignment different from the first relative alignment. The instructions may further include recoupling to the tube 102 the at least one of the first end cap or second end cap that was removed at the second relative alignment. If one or both of the first end cap 108 or second end cap 110 is an end cap assembly 200, the instructions may further include removing at least one fastener 214 from the UV generator 100 by removing the at least one fastener 214 from the at least one boss 216 on the tube 102 of the UV generator 100 and from the end cap assembly 200. For an end cap assembly 200, this would include removing the at least one fastener 214 that is inserted through the at least one boss 216, but not necessarily removing the at least one fastener 214 that is inserted through the third end cap portion 206. This allows for the third end cap portion 206 and the second end cap portion 204 to remain coupled together while also decoupling the still-coupled third end cap portion 206 and the second end cap portion 204 from the UV generator 100. This also may decouple the first end cap portion 202 from both the UV generator 100 and the still-coupled third end cap portion 206 and second end cap portion 204. Alternatively, the at least one fastener 214 that is inserted through the third end cap portion 206 may also be removed so as to decouple the third end cap portion 206 from the second end cap portion 204.

The instructions may further include recoupling the at least one fastener 214 to the first end cap portion 202, to the still-coupled second end cap portion 204 and third end cap portion 206 (or decoupled in the alternative), and to the at least one boss 216 of the UV generator 100. The instructions may further include rotating the second end cap portion 204, or the still-coupled second end cap portion 204 and third end cap portion 206, that was removed relative to the central axis B before recoupling back to the UV generator 100, such as providing instructions to rotate the second end cap portion 204, or the still-coupled second end cap portion 204 and third end cap portion 206, that was removed 0°, or 90°, or 180°, or 270° relative to the central axis B before recoupling back to the UV generator 100.

The method may further include following the instructions to adjust the UV generator 100 from a first relative alignment to a second relative alignment. The method may further include removing at least one of the first end cap 108 or second end cap 110 from a tube 102 of the UV generator 100. If one or both of the first end cap 108 or second end cap 110 is an end cap assembly 200, removing at least one of the first end cap 108 or second end cap 110 may include removing at least one fastener 214 from the UV generator 100 by removing the at least one fastener 214 from the at least one boss 216 on the tube 102 of the UV generator 100 and from the end cap assembly 200. For an end cap assembly 200, this would include removing the at least one fastener 214 that is inserted through the at least one boss 216, but not necessarily removing the at least one fastener 214 that is inserted through the third end cap portion 206. This allows for the third end cap portion 206 and the second end cap portion 204 to remain coupled together while also decoupling the third end cap portion 206 and the second end cap portion 204 from the UV generator 100. This also may decouple the first end cap portion 202 from both the UV generator 100 and the still-coupled third end cap portion 206 and second end cap portion 204. Alternatively, the at least one fastener 214 that is inserted through the third end cap portion 206 may also be removed so as to decouple the third end cap portion 206 from the second end cap portion 204.

The method may further include rotating the second end cap portion 204, or the still-coupled second end cap portion 204 and third end cap portion 206, that was removed relative to the central axis B, such as rotating the second end cap portion 204, or the still-coupled second end cap portion 204 and third end cap portion 206, that was removed 0°, or 90°, or 180°, or 270° (90 ° from the other direction) relative to the central axis B.

The method may further include securing the at least one of the first end cap 108 or second end cap 110 that was removed to the UV generator 100 in a second relative alignment that is different from the first relative alignment. For example, the method may include recoupling to the tube 102 the at least one of the first end cap 108 or second end cap 110 that was removed at the second relative alignment, such as recoupling the coupled third end cap portion 206 and second end cap portion 204 that was removed to the tube 102. If one or both of the first end cap 108 or second end cap 110 is an end cap assembly 200, recoupling the at least one of the first end cap 108 or second end cap 110 that was removed to the UV generator 100 in a second relative alignment may include recoupling the at least one fastener 214 to the first end cap portion 202, to the still-coupled second end cap portion 204 and third end cap portion 206 (or decoupled in the alternative), and to the at least one boss 216 of the UV generator 100.

In some non-limiting embodiments, the end cap assembly 200 may include an o-ring seal 212, such as a plurality of o-ring seals 212. The end cap assembly 200 may include at least one o-ring seal 212 positioned between at least one of the plurality of end cap portions. For example, the end cap assembly 200 may include an o-ring seal 212 between each of the end cap portions. The end cap assembly 200 may include a first o-ring seal 212 positioned between the first end cap portion 202 and the second end cap portion 204, and a second o-ring seal 212 positioned between the second end cap portion 204 and the third end cap portion 206. The combination of o-ring seals 212 and fasteners 214 to secure the end cap assembly 200 has the added benefit of applying compression directly to the o-ring seals 212 in a static crush arrangement (as shown in FIG. 5B), thereby forming a robust chamber sealing arrangement. Additionally, the fastener 214 arrangement also provides for a contiguous metal reinforcing structure for the end cap assembly 200, forming a metallic “skeleton” through the end cap assembly 200 that is predominately formed of plastic, or other non-metallic material(s). The metal skeleton formed from the fastener 214 arrangement provides added strength to the end cap assembly 200 and protects against failure of the end cap portions 202, 204, 206, while still enabling the various pieces of the end cap assembly 200 to be individually removed for maintenance purposes. The multi-piece arrangement of the end cap assembly 200 also simplifies the molding of each plastic end cap portion 202, 204, 206, thereby reducing the tooling complexity for forming the parts. This reduction in complexity may reduce the chance of quality issues associated with the molding process, and also reduces the cost of the tooling itself. Additionally, the cost of the overall end cap assembly 200 may be reduced as compared to a unitary molded end cap due to a reduced overall size and volume of material.

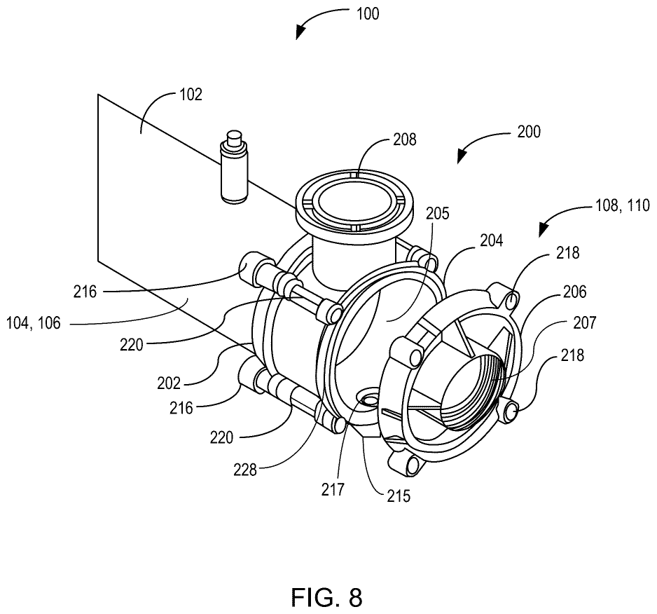

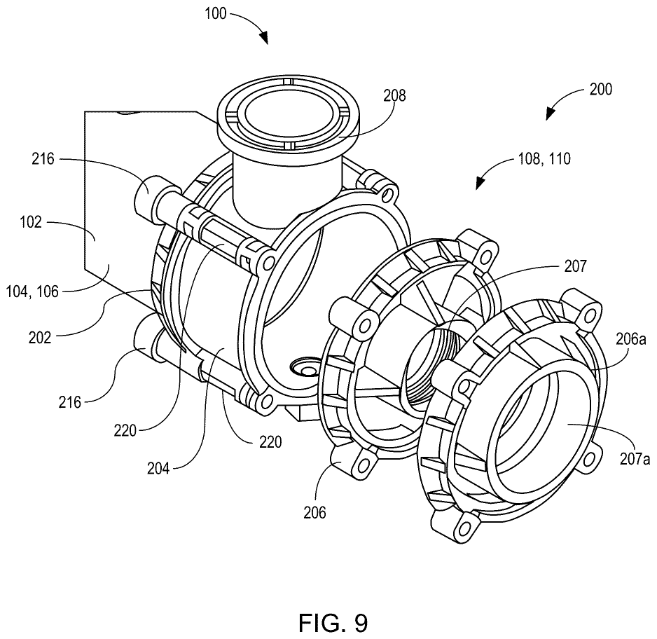

The multi-piece arrangement of the end cap assembly 200 also allows for easier maintenance of the UV generator 100 as a whole, as fewer components of the end cap assembly 200 need be removed to gain access to the internal surface 121 of the UV generator 100, including the process connection to a customer's pipework, as shown in FIGS. 8 and 9. Additionally, the multi-piece arrangement of the end cap assembly 200 more easily allows for future design changes and/or updates. For example, if a desired size or fitting of a UV lamp 116 to be used in the UV generator 100 were to be changed in the future, fewer pieces of the multi-piece assembly 200 (in some cases only one piece) would need to be changed to accommodate the use of such an alternate UV lamp 116. Similarly, if quality issues were to arise with certain parts of the assembly, that part (or those parts) can be individually removed and replaced, thereby saving time and cost in servicing the UV generator 100.

The end cap assembly 200 may allow for access to the internal chamber 123 of the tube 102 without removing the full end cap assembly 200. For example, the fastener(s) 214 that are positioned through the third end cap portion 206 can be removed while not removing the fastener(s) 214 that are positioned through the boss(es) 216. This allows for the third end cap portion 206 to be removed while maintaining the first end cap portion 202 and second end cap portion 204 coupled, such as fastened, to the tube 102. Removal of this third end cap portion 206 allows for access into the internal chamber 123 of the tube 102. While the third end cap portion 206 is removed from the end cap assembly 200, the third end cap portion 206 can be replaced with a new third end cap portion 206a, as shown in FIG. 9. The new third end cap portion 206a may be the same as the third end cap portion 206 except that the inner circumference of the opening 207a may be larger or smaller than the opening 207 of the third end cap portion 206 to account for a different size UV lamp 116 and/or secondary tube 125.

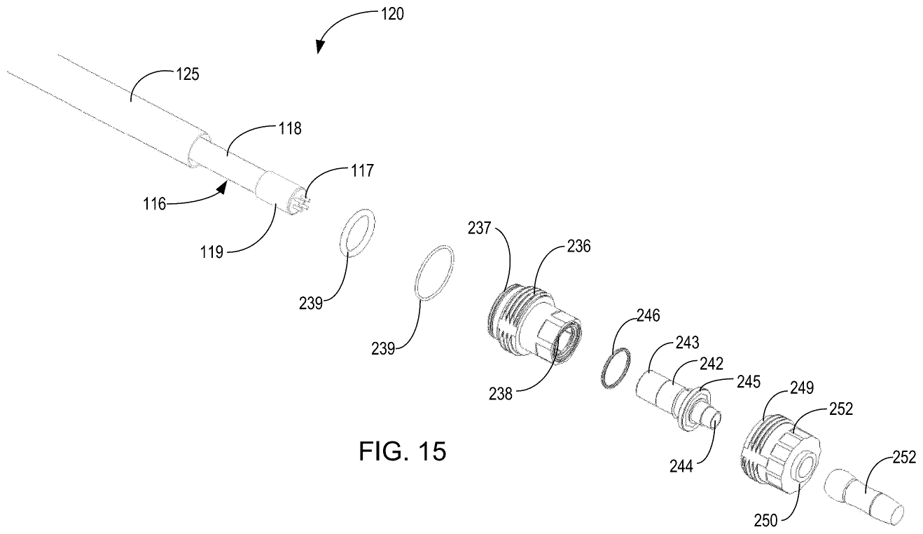

As shown in FIGS. 15 and 20-21, the UV generator 100 may include a sealing arrangement 120 for sealing tube 102, the secondary tube 125, and UV lamp 116. The sealing arrangement 120 may be a wedge sealing arrangement and/or method used for quartz sleeves and chamber bodies. Such wedge sealing arrangements and/or methods are shown in U.S. Pat. No. 10,541,503B2 , the disclosure of which is hereby incorporated by reference in its entirety. The sealing arrangement 120 may include a clamp ring 236. A first end 237 of the clamp ring 236 may be configured to be received by the opening 207 of the third end cap portion 206 and a second end 238 of the clamp ring 236 may be configured to receive a connector 242. The first end 237 of the clamp ring 236 may be externally threaded, and the second end 238 of the clamp ring 236 may be internally threaded. The clamp ring 236 may be coupled to an end of the secondary tube 125 and configured to secure the secondary tube 125 in the internal chamber 123 of the tube 102. The inner and/or outer circumference of the first end 237 of the clamp ring 236 may be different from the inner and/or outer circumference of the second end of the clamp ring 236. For example, the inner circumference of the first end 237 of the clamp ring 236 may be greater than the outer circumference of the secondary tube 125, such that the first end 237 of the clamp ring 236 may be positioned over and coupled to the secondary tube 125. The outer circumference of the first end 237 of the clamp ring 236 may be less than the circumference of the opening 207 of the third end cap portion 206, such that the first end 237 of the clamp ring 236 may be positioned in the opening 207 of the third end cap portion 206. The inner circumference of both the first end 237 and second end 238 of the clamp ring 236 may be greater than the outer circumference of the UV lamp 116, such that the UV lamp 116 may be removed from the internal chamber 123 of the tube 102 by removing the UV lamp 116 through the clamp ring 236. The first end 237 of the clamp ring 236 may be tapered, such as tapered at a 45° angle. Alternatively, the first end 237 of the clamp ring 236 may be flat, such that it is not tapered.

The sealing arrangement 120 may include a primary o-ring seal 239. The primary o-ring seal 239 may have an outer circumference that is less than the inner circumference of the first end 237 of the clamp ring 236 such that the primary o-ring seal may be positioned within the first end 237 of the clamp ring 236. The inner circumference of the primary o-ring seal 239 may be greater than the outer circumference of the secondary tube 125, such that the primary o-ring seal 239 may be positioned around the secondary tube 125. The primary o-ring seal 239 thereby provides a seal between the clamp ring 236 and the secondary tube 125. The sealing arrangement may include a secondary o-ring seal 240. The secondary o-ring seal 240 may have an outer circumference that is less than the inner circumference of the opening 207 of the third end cap portion 206, such that the secondary o-ring seal 240 may be positioned in the opening 207 of the third end cap portion 206. The inner circumference of the secondary o-ring seal 240 may be greater than the outer circumference of the first end 237 of the claim ring 236, such that the secondary o-ring seal 240 may be positioned around the first end 237 of the clamp ring 236. The secondary o-ring seal thereby provides a seal between the clamp ring 236 and the opening 207 of the third end cap portion 206.

The sealing arrangement may include a connector 242. In some non-limiting embodiments, the connector 242 a connecter as disclosed in U.S. Pat. No. 8,944,840B2 , the disclosure of which is hereby incorporated by reference in its entirety. A first end 243 of the connector 242 may have an outer circumference that is less than the inner circumference of both the first end 237 and second end 238 of the clamp ring 238 and the inner circumference of the secondary tube 125, such that the connector 242 may be at least partially positioned in the clamp ring 236 and the secondary tube 125. The first end 243 of the connector 242 may be coupled to the UV lamp 116. For example, the first end 243 of the connector 242 may be coupled, such as electrically coupled, to the electrical connections 117 of the UV lamp 116. A second end 244 of the connector may be positioned outside the clamp ring 236 and secondary tube 125 via the ridge 245 which interfaces with the second end 238 of the clamp ring 236. In some non-limiting embodiments, the sealing arrangement 120 may include a tertiary o-ring seal 246. The tertiary o-ring seal 246 may be positioned between the clamp ring 236 and the connector 242 and provide a seal between the ridge 245 and the second end 238 of the clamp ring 236.

The sealing arrangement 120 may include a clamp ring retainer 248. A first end 249 of the clamp ring retainer 248 may be configured to be received by the opening 207 of the third end cap portion 206 and a second end 250 of the clamp ring retainer 248 may be configured to receive a second end 244 of the connector 242. The outer circumference of the first end 249 of the clamp ring retainer 248 may be less than circumference of the opening 207 of the third end cap portion 206, such that the clamp ring retainer 248 may be positioned in the opening 207. The first end 249 of the clamp ring retainer 248 may be threaded. The inner circumference of the first end 249 of the claim ring retainer 248 may be greater than the outer circumference of the connector 242 including the ridge 245 and the outer circumference of the second end 238 of the clamp ring 236, such that the second end 244 and ridge 245 of the connector 242 may pass within the clamp ring retainer 248. The inner circumference of the second end 250 of the clamp ring retainer 248 may be greater than the outer circumference of the second end 244 of the connector 242 but less than the circumference of the ridge 245, such that the at least a portion of the second end 244 of the connector 242 while the ridge 245 interfaces with an inner surface of the second end 250 of the clamp ring retainer 248. The clamp ring retainer 248 may be configured to secure the connector 242 and the UV lamp 116 in the internal chamber 123 of the tube 102.

The sealing arrangement 120 may include a plug 252. In some non-limiting embodiments, the plug 252 may be a plug as disclosed in U.S. Pat. No. 8,944,840B2 . The plug 252 may have a size larger than the inner circumference of the second end 250 of the clamp ring retainer 248 such that the plug 252 interfaces with the clamp ring retainer 248 but does not pass into it. The plug 252 may include a cutout 253 configured to receive the second end 244 of the connector 242. The plug 252 may be configured to protect the second end 244 of the connector 242 until the second end 244 is ready to be coupled to an external electrical connection.

The sealing arrangement 120 of the present invention has the added benefit of allowing the UV lamp 116 and/or secondary tube 125 to be removed and/or replaced without removing the end cap assembly 200. For example, a method of removing and/or replacing the UV lamp 116 may include decoupling the clamp ring retainer 248 from the opening 207 of the third end cap portion 206. The method may further include decoupling the connector 242 from the UV lamp 116. The method may further include removing the UV lamp 116 from the internal chamber 123 of the tube 102 by removing the UV lamp 116 through the opening 207 of the third end cap portion 206 and through the clamp ring 236. The method may also include removing the plug 242 from the second end 244 of the connector 242 prior to decoupling the clamp ring retainer 248. A new UV lamp 116 may then replace the old one by inserting the UV lamp 116 through the opening 207 and the clamp ring 236, coupling the connector 242 to the electrical connections 117 of the UV lamp 116, and coupling the clamp ring retainer 248 back into the opening 207 of the third end cap portion 206. The plug 252 may also be coupled again to the second end 244 of the connector 242. In this regard, a method of installing one or more UV lamps 116 in the UV generator 100 is provided. The method may include providing instructions to install one or more UV lamps 116 in the UV generator 100, such as instructions including the aforementioned steps of removing a UV lamp 116 in reverse. The method may further include inserting a UV lamp 116 through the opening 207 of the third end cap portion 206 and into the internal chamber 123 of the UV generator 100. The method may further include coupling the connector 242 to the UV lamp 116, such as coupling the connector 242 to the electrical connections 117 of the UV lamp 116. The method may further include coupling the clamp ring retainer 248 to the opening 207 of the third end cap portion 206. The method may further include coupling the plug 252 to the second end 244 of the connector 242.

A method of removing and/or replacing the secondary tube 125 may include the same steps as the method of removing and/or replacing the UV lamp 116. The method of removing and/or replacing the secondary tube 125 may further include decoupling the clamp ring 236 from the opening 207 of the third end cap portion 206. The method may further include removing the secondary tube 125 from the internal chamber 123 of the tube 102 by removing the secondary tube 125 through the opening 207 of the third end cap portion 206. A new secondary tube 125 may then replace the old one by inserting the secondary tube 125 through the opening 207 into the internal chamber 123 of the tube 102 and then coupling the clamp ring 236 back into the opening 207.

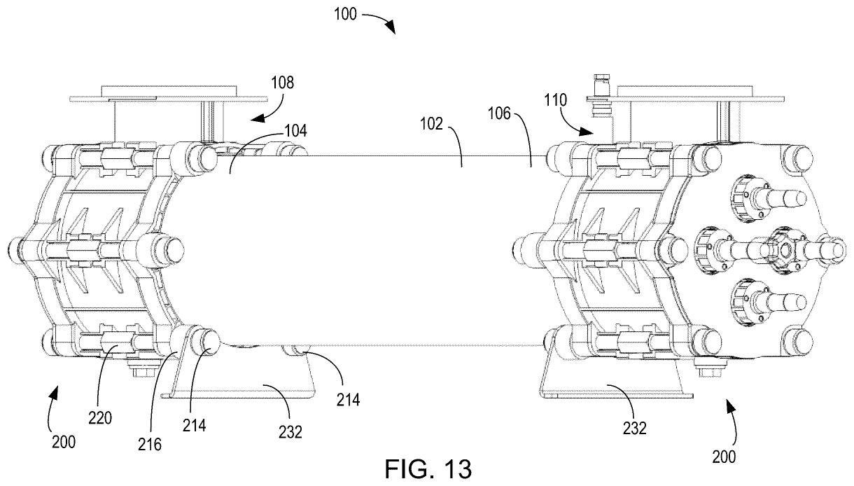

As shown in FIG. 13, in some non-limiting embodiments, the UV generator 100 may include a mounting bracket 232. The UV generator 100 may include at least two mounting bracket 232, with at least one mounting bracket 232 positioned at least end 104, 106 of the tube 102. The mounting bracket(s) 232 may include at least one bracket hole 234, such as a plurality of bracket holes 234. The position and spacing of the bracket holes 234 on the mounting bracket(s) 232 may correspond to a position and spacing of the holes 218 of the end cap assembly portions 202, 204, 206. In this regard, the mounting bracket(s) 232 may be fastened to the UV generator 100 may passing the fastener(s) 214 through the bracket hole(s) 234, thereby making the mounting bracket(s) 232 a part of the fastening structure of the end cap assembly 200. The mounting bracket 232 may include additional bracket holes 234 for mounting and attaching the UV generator 100 to an external surface.

In some non-limiting embodiments, a UV generator 100 may include a plurality of UV lamps 116, such as the UV generator 100 of FIGS. 16A-23. For example, the UV generator may include at least two, or at least three, or at least four UV lamps 116. The UV generator 100 may include a plurality of secondary tubes 125 such that each UV lamp 116 of the plurality of UV lamps 116 has a corresponding secondary tube 125. The UV generator 100 may include a plurality of sealing arrangements 120 such that each UV lamp 116 of the plurality of UV lamps 116 has a corresponding sealing arrangement 120. If the UV generator 100 is to include a plurality of UV lamps 116, the tube 102 may be enlarged to properly house the plurality of UV lamps 116. The end cap assembly 200 may also be enlarged to account for the larger size of the tube 102. The number of holes 218 on each portion 202, 204, 206 and the number of bosses 216 may be increased to provide a stronger fastening arrangement for the larger UV generator 100. For example, the tube 102 may include at least four bosses 216, or at least five bosses 216, or at least six bosses 216. The first end cap portion 202 and third end cap portion 206 may include at least four holes 218, or at least five holes 218, or at least six holes 218. The second end cap portion 204 may include at least eight holes 218, or at least ten holes 218, or at least twelve holes, split between the two ends of the second end cap portion 204. The third end cap portion 206 of the end cap assembly may include a plurality of openings 207 such that each UV lamp 116 of the plurality of UV lamps 116 has a corresponding opening 207 of the third end cap portion 207 to be inserted into and removed from.

Referring to FIG. 16B, the UV generator 100 may include a wiper arrangement 254. The wiper arrangement 254 may be automatic or manual, and configured to clean the hollow conduit 118 of the UV lamp(s) 116, the secondary tube(s) 125, and/or the internal surface 121 of the tube 102. The wiper arrangement 254 may include at least one wiper ring structure 256 including at least one wiper ring 258. In some non-limiting embodiments, the wiper arrangement 254 may include a plurality of wiper ring structures 256 so that each wiper ring structure 256 only has to clean a portion of the hollow conduit 118 of the UV lamp 116 or the secondary tube 125. For example, the wiper arrangement 254 may include at least two wiper ring structures 256. Each wiper ring structure 256 may include a plurality of wiper rings 258, corresponding to the number of UV lamps 116 present in the UV generator 100. The wiper ring(s) 258 may include a rubber layer 260 on the inner surface of the wiper ring(s) 258 that contacts the hollow conduit 118 of the UV lamp(s) 116 or the secondary tube(s) 125. Each wiper ring structure 256 may be attached to a rod 262 which spans the length of the tube 102. Alternatively, the wiper ring structure 256 may be replaced by a structure that includes a plurality of metal wire brushes for cleaning the hollow conduit 118 of the UV lamp 116 or the secondary tube 125. The rod 262 may be slidable relative to the tube 102 and UV lamps 116, such that the wiper ring(s) 258 may pass back and forth over the hollow conduit 118 of the UV lamp 116 or the secondary tube 125. If the wiper arrangement 254 is manual, the wiper arrangement 254 may include a handle 264 such that a user may operate the wiper arrangement 254.

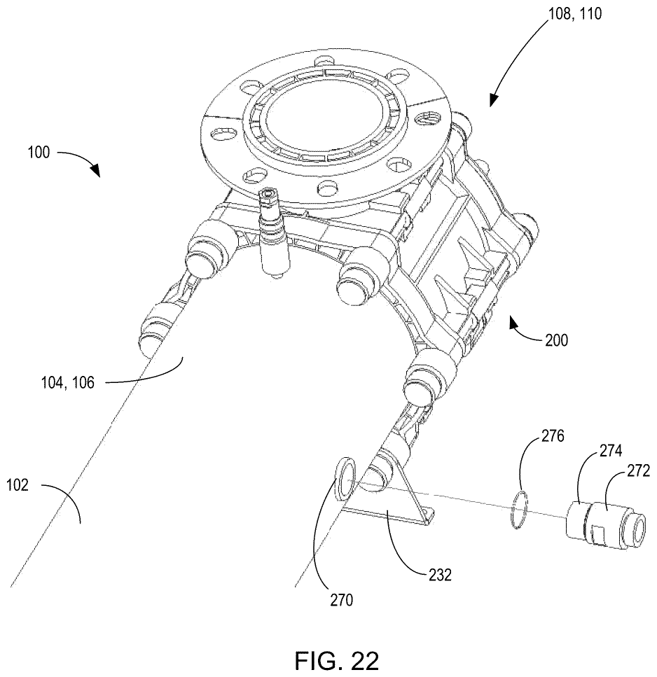

As shown in FIGS. 22-23, a UV sensor 112 may be positioned on the tube 102 and in a sensor port 270. A sensor window housing 272 may be positioned in the sensor port 270. The sensor window housing 272 may be configured to house the UV sensor 112 and provide a viewing window such that the UV sensor 112 can be seen within the sensor window housing 272. A sensor o-ring seal 276 may be positioned around a first end 274 of the sensor window housing 272 to provide a seal between the sensor port 270 and the sensor window housing 272. A first end 278 of the UV sensor 112 may be positioned in the sensor window housing 272 and a second end 280 of the UV sensor 112 may be positioned outside of the sensor window housing 272. A ridge 282 of the UV sensor 112 may interface with the sensor window housing 272. A sensor cap 284 may be positioned over the second end 280 of the UV sensor 112 and coupled to the sensor window housing 272 to secure the UV sensor 112 in the sensor port 270.

As shown in FIG. 24, in some non-limiting embodiments, a system 300 for fluid inactivation or sterilization may include a tank or other reaction vessel 302 for holding or circulating a fluid 304 to be treated with a UV radiation source. The fluid 304 in the tank or reaction vessel 302 may be transported to the tank or reaction vessel 302 from a different tank or reaction vessel. The UV generator 100 may be positioned within the tank and/or other reaction vessel 302, such that the fluid 304 in the vessel may be circulated through the UV generator 100 and exposed to a sufficient dose of the UV radiation. The inactivated/sterilized fluid 304 may then be transported external from the tank or reaction vessel, such as to another tank or reaction vessel for a different process or to a filter.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An ultraviolet (UV) generator, comprising:
a tube forming a main body of the UV generator, wherein the tube is comprised of titanium;
a first end cap assembly coupled to a first end of the tube, wherein the first end cap assembly is formed of a first plurality of end cap portions;
a second end cap assembly coupled to a second end of the tube opposite the first end, wherein the second end cap assembly is formed of a second plurality of end cap portions; and
at least one UV lamp extending between the first end cap assembly and the second end cap assembly and positioned within an internal chamber of the tube.

2. The UV generator of claim 1, wherein at least one portion of each of the respective first and second end cap assemblies is removable relative to one or more other portions of the respective first and second end cap assemblies.

3. The UV generator of claim 1, wherein each of the first end cap assembly and the second end cap assembly are coupled to the tube by one or more fasteners.

4. The UV generator of claim 3, wherein the tube comprises at least one boss extending from an outer surface thereof, and wherein the at least one boss is configured to receive the at least one fastener.

5. The UV generator of claim 1, further comprising at least one O-ring seal positioned between at least one of the plurality of end cap portions of each of the first end cap assembly and the second end cap assembly.

6. The UV generator of claim 1, wherein at least one of the first end cap assembly and the second end cap assembly comprises:
a first end cap portion coupled to the tube;
a second end cap portion comprising an inlet/outlet opening and coupled to the first end cap portion; and
a third end cap portion coupled to the second end cap portion.

7. The UV generator of claim 6, wherein the inlet/outlet opening of the second end cap portion is flush with the internal chamber of the tube.

8. The UV generator of claim 6, wherein the third end cap portion is removable relative to the first end cap portion and the second end cap portion.

9. The UV generator of claim 6, wherein the third end cap portion comprises at least one opening configured to allow the at least one UV lamp to be removed from the internal chamber of the tube through the at least one opening of the third end cap portion.

10. The UV generator of claim 1, further comprising at least one secondary tube comprised of quartz, the at least one secondary tube configured to surround the at least one UV lamp and extend between the first end cap assembly and the second end cap assembly, and positioned within an internal chamber of the tube.

11. The UV generator of claim 1, wherein at least one of the first end cap assembly and the second end cap assembly is at least partially rotatable relative to the tube.

12. The UV generator of claim 11, wherein the first end cap assembly is rotated 90° relative to the second end cap assembly.

13. The UV generator of claim 11, wherein the first end cap assembly is rotated 180° relative to the second end cap assembly.

14. The UV generator of claim 1, wherein the first end cap assembly is formed of at least one plastic material and the second end cap assembly is formed of at least one plastic material.

15. The UV generator of claim 14, wherein at least one of the first end cap assembly and the second end cap assembly are formed of at least one of high density polyethylene (HDPE), chlorinated polyvinyl chloride (CPVC), or polypropylene (PP).

16. The UV generator of claim 1, wherein the UV generator is configured to be mountable both vertically and horizontally.

17. The UV generator of claim 1, wherein an interior surface of the tube includes an interior coating.

18. The UV generator of claim 17, wherein the interior coating comprises a titanium dioxide coating.

19. The UV generator of claim 1, wherein the at least one UV lamp comprises a plurality of UV lamps extending between the first end cap assembly and the second end cap assembly.

20. The UV generator of claim 19, further comprising a plurality of secondary tubes comprising quartz, wherein each secondary tube surrounds a respective one of the plurality of UV lamps and extends between the first end cap assembly and the second end cap assembly.

21. A method, comprising:
providing a UV generator comprising a first end cap and a second end cap, wherein the first end cap and second end cap are at a first relative alignment, and one of the first end cap and second end cap is fluidly connectable to a source of fluid to be treated; and
providing instructions to adjust the first relative alignment to a second relative alignment by:
removing at least one of the first end cap or second end cap from a tube of the UV generator; and
securing to the tube the at least one of the first end cap or second end cap that was removed at a second relative alignment different from the first relative alignment.

22. The method of claim 21, further comprising instructions to install one or more UV lamps in the UV generator.

23. The method of claim 21, wherein the instructions further comprise:
recoupling to the tube the at least one of the first end cap or second end cap that was removed at the second relative alignment.

24. The method of claim 21, wherein the instructions further comprise:
rotating the at least one of the first end cap or second end cap relative to a central axis of the UV generator.

25. The method of claim 24, wherein the instructions further comprise:
rotating the at least one of the first end cap or second end cap 90° relative to the central axis.

26. The method of claim 24, wherein the instructions further comprise:
rotating the at least one of the first end cap or second end cap 180° relative to the central axis.

27. The method of claim 21, wherein at least one of the first end cap or second end cap comprises an end cap assembly.

28. The method of claim 27, wherein the instructions further comprise:
removing at least one fastener from at least one boss on the tube of the UV generator and from the end cap assembly.

29. The method of claim 27, wherein the instructions further comprise:
removing a coupled second end cap portion and third end cap portion of the end cap assembly from the tube and from a first end cap portion of the end cap assembly.

30. The method of claim 29, wherein the instructions further comprise:
recoupling the coupled second end cap portion and third end cap portion of the end cap assembly to the tube and to the first end cap portion in the second relative alignment.

31. A method, comprising:
providing a UV generator comprising a first end cap and a second end cap, wherein at least one of the first end cap and second end cap are at least partially rotatable relative to a central axis of the UV generator, the first end cap is fluidly couplable to a first pipe, and the second end cap is fluidly couplable to a second pipe; and
reconfiguring alignment of at least one of the first end cap and the second end cap by rotating at least one of the first end cap and the second end cap based on a position of at least one of the first pipe and the second pipe.
